(19) United States
(12) Patent Application Publication
(10) Pub. No.: US 2016/0194425 A1
(43) Pub. Date: Jul. 7, 2016

(54) HIGHLY EXPANDABLE HYDROGELS IN MEDICAL DEVICE SEALING TECHNOLOGY

(71) Applicant: Endoluminal Sciences Pty. Ltd., Sydney (AU)

(72) Inventors: Ashish Sudhir Mitra, Sydney (AU); Jens Sommer Knudsen, East Killara (AU); Roya Ravarian, Artarmon (AU); Cristina Borras Guardiola, Sydney (AU); Pak Man Victor Wong, Leichhardt (AU)

(21) Appl. No.: 14/987,459

(22) Filed: Jan. 4, 2016

Related U.S. Application Data
(60) Provisional application No. 62/099,769, filed on Jan. 5, 2015.
(60) Provisional application No. 62/165,023, filed on May 21, 2015.

(51) Int. Cl. C08F 222/38; C08K 5/053; A61L 31/16; C08K 5/1545; A61L 31/04; A61L 31/14; A61F 2/07; C08K 5/1535

(52) U.S. Cl. CPC C08F 222/385; A61F 2/07; C08K 5/053; C08K 5/1535; C08K 5/1545; A61L 31/048; A61L 31/145; A61L 31/16; A61F 2002/077; A61F 2220/0008; A61L 2400/06; A61L 2400/04; A61L 2300/418

(57) ABSTRACT

Highly expandable materials have been developed for filling an aneurysm sac and for sealing of endoluminal devices vessel walls. The expandable materials have appropriate chemical and physical properties to withstand radiation, sterilization, or storage in sterilizing solution, without loss of expandable characteristics. The expandable materials may contain protectants, prophylactic, diagnostic, therapeutic, or imaging agents. The expandable materials form a seal that actively conforms to vascular anatomy sealing any leaks that may occur after device implantation. In one embodiment, the technology is used to prevent leaks associated with abdominal aortic aneurysm (AAA) repair, especially for complex AAA repair.

### CROSS REFERENCE TO RELATED APPLICATIONS

This application claims priority to and benefit of U.S. Provisional Application No. 62/099,769 “Highly Expandable Hydrogels” filed on Jan. 5, 2015, and U.S. Provisional Application No. 62/165,023 “Sealing Technology for Treatment of Complex AAA” filed on May 21, 2015, the disclosures of which are hereby incorporated herein by reference in their entirety.

### FIELD OF THE INVENTION

The present disclosure is directed generally to highly expandable hydrogels and their use as sealing means for sealing endoluminal devices to vessel walls or as filling means for aneurysm sac filling.

### BACKGROUND OF THE INVENTION

An aneurysm is a localized, blood-filled dilation of a blood vessel caused by disease or weakening of the vessel wall. Aneurysms affect the ability of the vessel to conduct fluids, and can be life threatening if left untreated. Aneurysms most commonly occur in arteries at the base of the brain and in the aorta. As the size of an aneurysm increases, there is an increased risk of rupture, which can result in severe hemorrhage or other complications including sudden death. Aneurysms are typically treated by surgically removing a part or all of the aneurysm and implanting a replacement prosthetic section into the body lumen. Such procedures, however, can require extensive surgery and recovery time. Patients often remain hospitalized for several days following the procedure, and can require several months of recovery time. Moreover, the morbidity and mortality rates associated with such major surgery can be significantly high.

Another approach for treating aneurysms involves remote deployment of an endovascular graft assembly at the affected site. Such procedures typically require intravascular delivery of the endovascular graft assembly to the site of the aneurysm. The graft is then expanded or deployed in situ and the ends of the graft are anchored to the body lumen on each side of the aneurysm. In this way, the graft effectively excludes the aneurysm sac from circulation.

One concern with many conventional endovascular graft assemblies, however, is the long term durability of such structures. Over time, the graft can become separated from an inner surface of the body lumen, resulting in bypassing of the blood between the vessel wall and the graft, causing endoleaks. When an endoleak occurs, it can cause continuous pressurization of the aneurysm sac and may result in an increased risk of rupture.

Endoleaks are classified into the following five types.

Type I endoleaks occur as a result of an inadequate seal at the site of the graft attachment. It may occur at the proximal end, distal end or where the components overlap. Blood flow leaks alongside the graft into the aneurysm sac. It occurs in as many as 10% of cases. They are often the result of unsuitable patient (aneurysm) selection or device selection, but can also occur if the graft migrates. Type I leaks are always considered significant as they do not tend to resolve spontaneously.

Type II endoleaks are the most common after an abdominal aortic repair, accounting for 80% of cases. Retrograde flow though branch vessels continues to fill the aneurysm sac. The most common culprit vessels are lumbar arteries, inferior mesenteric artery or internal iliac artery. This type of leak has been reported in up to 25% of cases. It usually resolves spontaneously over time and requires no treatment. Embolization of the branch vessel is indicated if the aneurysm sac continues to expand in size.

Type III endoleaks are caused by mechanical failure of the stent-graft. There may be a fracture of the stent-graft, hole or defect on the graft fabric, or junctional separation of the modular components. Causes may relate to defective device material, extreme angulation of a segment predisposing to fracture, or improper overlap of the modular components during insertion.

Type IV endoleaks occur when blood leaks across the graft due to its porosity. It does not require any treatment and typically resolves within a few days of graft placement.

Type V “leak” (also referred to as endotension) is not a true leak but is defined as continued expansion of the aneurysm sac without evidence of a leak site. It is also referred to as endotension. It is a poorly understood phenomenon but is believed to be due to pulsation of the graft wall with transmission of the pulse wave through the perigraft space (aneurysm sac) to the native aneurysm wall.

Embolization of aneurysms with tissue fillers, including hydrogels, is described in U.S. Pat. Nos. 7,790,194, 8,465,779, 8,231,890, and in U.S. Publication No. US 2014/0228453.

Embolization devices with expandable compositions for sealing aneurysms are also known in the art. Examples of various embolization devices are described in U.S. Pat. Nos. 8,313,504, 8,083,768, 8,231,890, 8,465,779, 8,814,928, and in U.S. Publication Nos. US 2007/0244544, US 2010/0131001, US 2012/0089218, US 2014/0052168, US 2014/0194973, US 2014/0228453. Commercially available embolization devices include AZUR™ Peripheral HydroCoil by Terumo, AXIUM™ Embolic Coils by Covidien, EV3 PIPELINE™ device by Covidien, and the OVATION PRIME™ system by TriVascular.

The concern with the available endovascular devices is inadequate sealing of the devices to vessel walls and the long-term stability and durability of the devices in situ (Baril et al., Ann. Vasc. Surg., 22(1):30-36, 2008). The devices known in the art are often unstable and may easily dislodge and/or migrate from the site of implantation.

There is still a need for compositions that may be used as embolizing agents, or as means for sealing an endoluminal device at the site of implantation, that provide adequate sealing and long-term stability.

It is therefore an object of the present invention to provide highly expandable materials that rapidly activate in situ, have sufficient pressure to secure but not deform or displace the implanted prosthesis, are biocompatible, retain strength and flexibility in situ over a prolonged period of time, and can seal endoleaks and/or prevent leaks at sites of implantation.

It is a further object of the present invention to provide highly expandable materials with appropriate chemical and physical properties to withstand radiation, sterilization, or storage in sterilizing solution without loss of expandable characteristics.

It is another object of the present invention to provide highly expandable materials for filling an aneurysm sac.

It is a further object of the present invention to provide expandable materials with the appropriate chemical and physical properties as sealing means to seal an endoluminal device to a vessel wall. The sealing means actively conform to the vascular anatomy if any remodeling occurs after implantation so that any resulting leaks are sealed.

### SUMMARY OF THE INVENTION

Highly expandable materials, capable of rapidly swelling and increasing the weight of their dry state between approximately two and one hundred-fold, have been developed. The expandable materials retain their swelling characteristics after radiation, sterilization, or storage in sterilizing or storage solution(s). The expandable materials expand rapidly and reach their maximum weight within minutes following exposure to an aqueous fluid, such as phosphate-buffered saline (PBS) or blood. In some embodiments, the expandable materials include protectants, prophylactic, diagnostic, therapeutic and/or imaging agents.

Generally, the expandable materials are hydrogels, foams or sponges. In preferred embodiments, the expandable materials are hydrogels. Preferably, the expandable materials contain acrylamide and/or acrylic acid monomers crosslinked with polyvalent crosslinking agents such as bis-acrylamide or di-acrylamide, poly(ethylene glycol) diacrylamide, di(ethylene glycol) diacrylate, poly(ethylene glycol) diacrylate, and long-chain hydrophilic polymers with multiple polymerizable groups. In some embodiments the expandable materials are co-polymers made from two or more types of monomers and/or cross-linkers. In a preferred embodiment the expandable material consists of a copolymer of acrylamide and acrylic acid monomers cross-linked with a cross-linker such as Bis-acrylamide.

In some embodiments, the expandable materials are composite hydrogels. Typically, the composite hydrogels are formed of pieces of two or more types of hydrogel placed in close proximity to one another. The composite hydrogels are encapsulated. In some embodiments, the composite hydrogels include any combination of poly(acrylic acid), poly(acrylamide), and poly(metacrylic acid), and copolymers and blends of each. In preferred embodiments, the composite hydrogels are formed of cross-linked poly(acrylic acid) and poly(acrylamide).

Generally, the expandable materials swell in aqueous fluid, increasing their weight relative to the dry state from two to 100 fold, typically from 20 to 90 fold, and preferably from 20 to 60 fold. Generally, the expandable materials swell and reach their maximum weight in less than about 120 minutes, typically, within less than about 60 minutes, and preferably, within less than about 15 minutes, after placement in aqueous fluid.

In some embodiments, the expandable materials contain protectants. Typically, the protectants include compounds such as glycerin and/or ascorbic acid. In preferred embodiments, the protectant is glycerin. Preferably, the addition of protectants does not affect the swelling characteristics of the hydrogels and protects the integrity of the hydrogels. The protectants “absorb” any free radicals that are produced when the hydrogel is exposed to ionizing radiation, thereby preventing any further crosslinking or other types of radiation damage such as chain scission. The expandable materials containing protectants are more flexible so that they conform to a circular prosthesis after drying, and are compatible with the crimping and loading process of the device, i.e. they do not break during the crimping and loading process. In preferred embodiments, the expandable materials containing protectants are easier to dry to a consistent thickness. The protectants make the drying process more robust by protecting against over-drying.

In preferred embodiments, the expandable materials do not swell when stored in storage or sterilization solution. A solution for storing and/or sterilizing may be an aqueous solution under conditions suitable for storing natural tissue. Suitable solutions for storage of the expandable materials and devices containing the expandable materials include water miscible solvents, organic and inorganic molecules and salts thereof. Solutions of water miscible organic and inorganic molecules, or their salts, suitable for storage and/or sterilization, include organic compounds glutaraldehyde, formaldehyde, ethanol, propanol, bactericidal or bacteriostatic formulations, fungicide formulations, polyethylene glycol (PEG), polypropylene glycol (PPG), copolymers of PEG and PPG, glycerol, ethanol, isopropanol, and other inorganic salts such as sodium chloride, sodium sulfate, and other salts that limit the swelling of the gel due to hydrophobic interactions and/or ionic interactions. The expandable materials do not change their swelling characteristics after storage in or washing with storage/sterilization solutions. In preferred embodiments, the storage/sterilization solution is 30-100% ethanol. In preferred embodiments, the expandable materials retain their swelling characteristics after sterilization with ethylene oxide.

The expandable materials may be used in or as sealing means for sealing endoluminal devices to vessel walls, or as filling means for lumens such as aneurysm sacs or potentially in diverticulitis. The sealing of endoluminal devices, or the filling of aneurysm sac, may be configured to retain blood flow through the device.

In preferred embodiments, the expandable materials are hydrogels that can be encapsulated into a seal. The seal includes a flexible component that is configured to conform to irregularities between the endoluminal device and a vessel wall. The seal can be composed of a permeable, semi-permeable, or impermeable material. It may be biostable or biodegradable. The seal may be provided in a variety of shapes, depending on the device it is to be used with.

Generally, the device used with the seal can be any endovascular device, or any medical device in need of attachment to wall of a body lumen. In one embodiment, the devices are suitable for use with abdominal or thoracic stent grafts. In another embodiment, the devices with the seal are used for abdominal aortic aneurysm (AAA) repair, especially for complex AAA repair. Suitable devices include, but are not limited to, bifurcated stent grafts and chimney EVAR devices.

In all embodiments, it is absolutely critical that the hydrogel/expandable material operates under sufficiently low pressure that it does not push the device away from the wall or alter the device configuration. These materials must expand quickly (less than 30 minutes, more preferably less than 15 minutes to full swelling) and retain the desired mechanical and physiochemical properties for an extended period of time, even under the stress of being implanted within the vasculature or heart. Gels having the desired mechanical and swellable properties have been developed, as demonstrated by the examples.

These devices have the advantages of providing excellent sealing in combination with a low profile, controlled or contained release, and active conforming to leak sites to eliminate prosthetic-annular incongruence. If vascular re-modeling occurs over time, which could lead to leakage, the seal will also remodel, preventing leaks from developing. For devices that are at high risk of leakage, a pleated or accordion-like design provides for even better coverage and prevents uneven distribution of seal filler.

### BRIEF DESCRIPTION OF THE DRAWINGS

FIG. 1A is a scatter plot showing short-term swelling profile in bovine serum of a low temperature control sample without radiation and in the absence of the protectant glycerine. Swelling profile is presented as swelling ratio (%) over time (minutes). FIG. 1B is a scatter plot showing long-term swelling profile in bovine serum of a low-temperature control sample without radiation and in the absence of the protectant glycerine. Swelling profile is presented as swelling ratio (%) over time (days).

FIG. 2A is a scatter plot showing short-term swelling profile in bovine serum of a low temperature control sample without radiation and containing the protectant glycerine. Swelling profile is presented as swelling ratio (%) over time (minutes). FIG. 2B is a scatter plot showing long-term swelling profile in bovine serum of a low-temperature control sample without radiation and containing the protectant glycerine. Swelling profile is presented as swelling ratio (%) over time (days).

FIG. 3A is a scatter plot showing short-term swelling profile in bovine serum of an ambient-temperature control sample without radiation and containing the protectant glycerine. Swelling profile is presented as swelling ratio (%) over time (minutes). FIG. 3B is a scatter plot showing long-term swelling profile in bovine serum of an ambient-temperature control sample without radiation and containing the protectant glycerine. Swelling profile is presented as swelling ratio (%) over time (days).

FIG. 4A is a scatter plot showing short-term swelling profile in bovine serum of a sample radiated with low temperature electron beam (E-beam) in the absence of the protectant glycerine. Swelling profile is presented as swelling ratio (%) over time (minutes). FIG. 4B is a scatter plot showing long-term swelling profile in bovine serum of a sample radiated with low temperature E-beam in the absence of the protectant glycerine. Swelling profile is presented as swelling ratio (%) over time (days).

FIG. 5A is a scatter plot showing short-term swelling profile in human serum of a sample radiated with low temperature E-beam and containing the protectant glycerine. Swelling profile is presented as swelling ratio (%) over time (minutes). FIG. 5B is a scatter plot showing long-term swelling profile in human serum of a sample radiated with low temperature E-beam and containing the protectant glycerine. Swelling profile is presented as swelling ratio (%) over time (days).

FIG. 6A is a scatter plot showing short-term swelling profile in human serum of a sample radiated with ambient temperature E-beam and containing the protectant glycerine. Swelling profile is presented as swelling ratio (%) over time (minutes). FIG. 6B is a scatter plot showing long-term swelling profile in human serum of a sample radiated with ambient temperature E-beam and containing the protectant glycerine. Swelling profile is presented as swelling ratio (%) over time (days).

FIG. 7A is a scatter plot showing short-term swelling profile in bovine serum of a sample radiated with low temperature E-beam and containing the protectant glycerine. Swelling profile is presented as swelling ratio (%) over time (minutes). FIG. 7B is a scatter plot showing long-term swelling profile in bovine serum of a sample radiated with low temperature E-beam and containing the protectant glycerine. Swelling profile is presented as swelling ratio (%) over time (days).

FIG. 8A is a scatter plot showing short-term swelling profile in bovine serum of a sample radiated with ambient temperature E-beam and containing the protectant glycerine. Swelling profile is presented as swelling ratio (%) over time (minutes). FIG. 8B is a scatter plot showing long-term swelling profile in bovine serum of a sample radiated with ambient temperature E-beam and containing the protectant glycerine. Swelling profile is presented as swelling ratio (%) over time (days).

FIG. 9A is a scatter plot showing short-term swelling profile in PBS of a sample radiated with low temperature E-beam and containing the protectant glycerine. Swelling profile is presented as swelling ratio (%) over time (minutes). FIG. 9B is a scatter plot showing long-term swelling profile in PBS of a sample radiated with low temperature E-beam and containing the protectant glycerine. Swelling profile is presented as swelling ratio (%) over time (days).

FIG. 10A is a scatter plot showing short-term swelling profile in PBS of a sample radiated with ambient temperature E-beam and containing the protectant glycerine. Swelling profile is presented as swelling ratio (%) over time (minutes). FIG. 10B is a scatter plot showing long-term swelling profile in PBS of a sample radiated with ambient temperature E-beam and containing the protectant glycerine. Swelling profile is presented as swelling ratio (%) over time (days).

FIG. 11A is a scatter plot showing the swelling profile in human serum during the first two hours of swelling of a sample sterilized by ethylene oxide in the absence of protectant glycerin. Swelling profile is presented as swelling ratio (%) over time (minutes). FIG. 11B is a scatter plot showing swelling profile in human serum during the first three weeks of a sample sterilized by ethylene oxide in the absence of protectant glycerin. Swelling profile is presented as swelling ratio (%) over time (days).

FIG. 12A is a scatter plot showing swelling profile during the first two hours of swelling in human serum of a sample sterilized by ethylene oxide and containing the protectant glycerine. Swelling profile is presented as swelling ratio (%) over time (minutes). FIG. 12B is a scatter plot showing swelling profile during the first three weeks of swelling in human serum of a sample sterilized by ethylene oxide and containing the protectant glycerine. Swelling profile is presented as swelling ratio (%) over time (days).

FIG. 13A is a scatter plot showing swelling profile during the first two hours of swelling in bovine serum of a sample sterilized by ethylene oxide and containing the protectant glycerine. Swelling profile is presented as swelling ratio (%) over time (minutes). FIG. 13B is a scatter plot showing swelling profile during the first three weeks of swelling in bovine serum of a sample sterilized by ethylene oxide and containing the protectant glycerine. Swelling profile is presented as swelling ratio (%) over time (days).

FIG. 14A is a scatter plot showing swelling profile during the first two hours of swelling in PBS of a sample sterilized by ethylene oxide in the absence of the protectant glycerine. Swelling profile is presented as swelling ratio (%) over time (minutes). FIG. 14B is a scatter plot showing swelling profile during the first three weeks of swelling in bovine serum of a sample sterilized by ethylene oxide in the absence of the protectant glycerine. Swelling profile is presented as swelling ratio (%) over time (days).

FIG. 15A is a scatter plot showing swelling profile during the first two hours of swelling in PBS of a sample sterilized by ethylene oxide and containing the protectant glycerine. Swelling profile is presented as swelling ratio (%) over time (minutes). FIG. 15B is a scatter plot showing swelling profile during the first three weeks of swelling in PBS of a sample sterilized by ethylene oxide and containing the protectant glycerine. Swelling profile is presented as swelling ratio (%) over time (days).

FIG. 16A is a scatter plot showing swelling profile in PBS during the first two hours of swelling of a sample before storage in 70% ethanol. Swelling profile is presented as swelling ratio (%) over time (minutes). FIG. 16B is a scatter plot showing swelling profile in PBS during the first two hours of swelling of a sample after storage in 70% ethanol. Swelling profile is presented as swelling ratio (%) over time (minutes).

FIG. 17 is a line graph showing the change in thickness of capsule (mm) over time (mins) during swelling of encapsulated single (1) and composite (2 and 3) gels in phosphate buffered saline (“PBS”).

FIGS. 18A and 18B are graphs of the swelling profile of acrylamide 95%-acrylic acid 5% copolymer hydrogels in PBS (FIG. 18A) and bovine serum (FIG. 18B). FIGS. 18C and 28D are graphs of the swelling profile of acrylamide 80%-acrylic acid 20% copolymer hydrogels in PBS (FIG. 18C) and bovine serum (FIG. 18D). FIGS. 18E and 18F are graphs of the swelling profile of acrylamide 60%-acrylic acid 40% copolymer hydrogels in PBS (FIG. 18E) and bovine serum (FIG. 18F).

FIG. 19 is a bar graph showing the change in surface area (mm²) of a copolymer hydrogel containing 40%-90% acrylamide after swelling in PBS and then storage in storage solution containing 50%-100% poly(ethylene glycol) containing 0.5-5 M sodium and phosphate salts.

FIGS. 20A and 20B are prospective view drawings showing sealing of a type II leak by hydrogel attached on the outside of a stent graft. FIG. 20C is a plan view sketch of hydrogel-deposited stent graft.

FIGS. 21A and 21B are prospective view drawings showing sealing of a type II leak by hydrogel attached on the contralateral limb (or an independent limb), and not on the main body, of a stent graft.

FIGS. 22-26 are drawings showing how the highly expandable hydrogels can be utilized to seal endoleaks in complex AAA.

FIG. 22 is a drawing showing the seal with an inner semi-permeable membrane for controlled activation of the hydrogel, and an outer membrane for controlled expansion and complete encapsulation of the hydrogel.

FIGS. 23A and 23B are drawings showing the seal in the unexpanded state attached to a stent graft. FIG. 23A shows three-dimensional front view and FIG. 23B shows a three-dimensional crossectional view of the seal attached to the stent graft. The dry hydrogel is deposited in a center of a capsule, and the capsule extends about the perimeter and on the outside of the stent graft. FIGS. 23C and 23D are drawings showing the seal in the expanded state when the hydrogel has expanded. When implanted into the vessel, the seal will be positioned between the vessel wall and the sent graft, sealing the graft to the vessel wall.

FIGS. 24A and 24B are drawings showing a bifurcated stent graft without the seal. When in use, such a graft generates a leak site due to incomplete apposition of the stent graft to a heavily thrombotic vessel wall (FIG. 24B). FIGS. 24C and 24D are drawings showing a bifurcated stent graft with an expandable seal. Inclusion of the seal onto the bifurcated stent graft prevents the leak site from forming, as the seal is highly conformable and remains securely positioned following expansion (FIGS. 24C and 24D).

FIGS. 25A-25C are drawings showing a simulated sealing of gutter leak sites with use of the seal during implantation of a ChEVAR device. The gutter leak sites are present before activation of the seal hydrogel (FIG. 25B), but are eliminated after the activation of the hydrogel (FIG. 25C).

FIG. 26 is a drawing showing the seal can be present on both the chimneys of the ChEVAR device, and the stent graft. Using such a device, the gutter leak sites can be further minimized, as shown in the enlarged segment on the right.

### DETAILED DESCRIPTION OF THE INVENTION

### I. Definitions

As used herein, “hydrogel” refers to a substance formed when an organic polymer (natural or synthetic) is crosslinked via covalent, ionic, or hydrogen bonds to create a three-dimensional structure, which entraps or bonds with water molecules or activating fluid, such as aqueous fluid.

As used herein, “biocompatible” generally refers to a material and any metabolites or degradation products thereof that are generally non-toxic to the recipient and do not cause any significant adverse effects to the subject.

As used herein, “biodegradable” generally refers to a material that will degrade or erode by hydrolysis or enzymatic action under physiologic conditions to smaller units or chemical species that are capable of being metabolized, eliminated, or excreted by the subject. The degradation time is a function of material composition and morphology.

As used herein, “endoleak” is defined as a persistent blood or other fluid flow outside the lumen of the endoluminal graft, but within the aneurysm sac or adjacent vascular segment being treated by the device.

As used herein, “rapidly expanding” refers to a material which reaches its desired dimensions in less than thirty minutes, preferably in less than twenty minutes after activation or exposure to fluid, more preferably in less than fifteen minutes, after activation or exposure to fluid.

As used herein, “expandable” or “swelling” refers to a property of the hydrogels to increase in weight and in volume when the hydrogels absorb fluid.

As used herein, the term “swelling ratio” means a ratio of the weight of a hydrogel in fluid to that in its dry state, (multiplied by 100).

As used herein, the term “speed of swelling” means a change in swelling ratio over time.

As used herein, the term “stability”, when referred to hydrogels, means absence of change in swelling ratio over time. The stability may be measured over time that may be minutes, hours, days, months, or years ranging from between 5 min to 3 years.

As used herein, the terms “swelling profile” and “swelling characteristics” means a combination of swelling properties of hydrogels, including speed of swelling and stability.

### II. Compositions

Expandable materials that swell in contact with an aqueous fluid are disclosed. The expandable materials expand and increase in weight from two to 100 times the weight of their dry state. Preferably, the expandable materials increase in weight from 10 to 90 times, and most preferably from 20 to 60 times.

The expandable material can be a hydrogel, a foam, or a sponge. In preferred embodiments, the material is a hydrogel and/or a composite hydrogel.

Blood and/or other fluids can penetrate into a dried expandable material, causing the dried expandable material to absorb the fluid and swell or react to expand due to formation or release of gas reaction products. By expanding, the material fills the endoluminal space.

The expandable materials are preferably stable at both room temperature and 37-40° C. and can be sterilizable by one or more means such as radiation, steam, or organic solvents. The expandable materials are preferably made from biocompatible materials that allow tissue ingrowth or endothelialisation of the matrix. Such endothelialisation or tissue ingrowth can be facilitated either through selection of appropriate polymeric materials or by including with the material suitable active agents, such as growth promoting factors or proteins.

Also disclosed are devices incorporating seals containing the rapidly expandable hydrogels, which are used to secure the devices and prevent leaks at the sites of implantation.

A. Hydrogels

Expandable gels have been developed that are stronger and more resilient than current expandable gels. The mechanical strength of the swollen hydrogels ranges from between 0.00005 N/mm² and 0.025 N/mm². The mechanical strength of the hydrogels may be any value within this range, including 0.00005 N/mm² and 0.025 N/mm². The hydrogels are able to retain their mechanical strength while in an activated (swollen) state. The hydrogels are also resilient as they are able to constrict to their dry state from the activated state, and then get activated again and adopt their mechanical strength. These gels are able to expand rapidly and increase in weight to at least 10×, 20×, 25×, 30, 40×, or 50×, or up to 60×, the weight of their dry state in less than 25, 24, 23, 22, 21, 20, 19, 18, 17, 16, 15, 14, 13, 12, 11, 10, 9, 8, 7, 6, 5, or 4 minutes when exposed to physiological liquids.

### 1. Suitable Hydrogel Components

Suitable components of such gels include, but are not limited to, acrylic acid, acrylamide or other polymerizable monomers; cross-linkers such as bis-acrylamide, poly(ethylene glycol) diacrylamide (PEGDAA), N,N′-methylenebisacrylamide, poly(ethylene glycol) diacrylate (PEGDA), polyvinyl alcohols as well as partially hydrolyzed poly vinyl acetates, 2-hydroxyethyl methacrylates (HEMA) or various other polymers with reactive side groups such as acrylic, allylic, and vinyl groups, can be used. In addition, a wide range of natural hydrocolloids such as dextran, cellulose, agarose, starch, galactomannans, pectins, hyaluronic acid etc. can be used. Reagents such as allyl glycidyl ether, allyl bromide, allyl chloride etc. can be used to incorporate the necessary double bonds to participate in a free radical polymerization reaction or addition reaction, such as those containing acrylic, allylic and vinyl groups, into the backbones of these polymers. Depending on the chemistry employed, a number of other reagents can be used to incorporate reactive double bonds.

Studies to identify hydrogels having substantial swelling in a short time were performed, as described in the examples. The main factors that influence swelling of a hydrogel based on polymerization and cross-linking of synthetic monomers are:

(1) type of monomer;

(2) type of cross-linker;

(3) concentration of monomer and cross-linker in the gel; and

(4) the ratio of monomer to cross-linker.

Examples of rapidly swelling hydrogels include, but are not limited to, acrylamide polymers, acrylic acid polymers, and copolymers, particularly crosslinked acrylamide polymers, crosslinked acrylic acid polymers, and copolymers. Suitable crosslinking agents include di-acrylamide or bis-acrylamide crosslinkers, poly(ethylene glycol) diacrylamide, di(ethylene glycol) diacrylate, poly(ethylene glycol) diacrylate, and long-chain hydrophilic polymers with multiple polymerizable groups, such as poly vinyl alcohol (PVA) derivatized with allyl glycidyl ether.

### a. Copolymers

Copolymers of acrylamide (AAM) and acrylic acid (AA) in various ratios of AAM to AA may be used to form hydrogels as expandable materials of the seal. The copolymers may be crosslinked by any suitable crosslinker to form the hydrogel. Suitable crosslinkers include, but are not limited to, di-acrylamide or bis-acrylamide crosslinkers, poly(ethylene glycol) diacrylamide, di(ethylene glycol) diacrylate, poly(ethylene glycol) diacrylate, and long-chain hydrophilic polymers with multiple polymerizable groups, such as poly vinyl alcohol (PVA) derivatized with allyl glycidyl ether.

The various ratios of AAM to AA may range from between 60 weight percent (%) AAM to 40 weight % of AA and 95 weight % of AAM to 5 weight % of AA. The copolymers may be formed with crosslinkers and one or more initiators, such as of initiators ammonium persulfate (APS), potassium persulfate, sodium persulfate, and N,N,N′,N′-tetramethylethylenediamine (TEMED). In some embodiments, one or more initiators may vary in concentration or be absent.

### b. Other Natural and Synthetic Polymers and Copolymers

Additional examples of materials which can be used to form a suitable hydrogel include polysaccharides such as alginate, polyphosphazines, poly(acrylic acids), poly(methacrylic acids), poly(alkylene oxides), poly(vinyl acetate), polyvinylpyrrolidone (PVP), and copolymers and blends of each. See, for example, U.S. Pat. Nos. 5,709,854, 6,129,761 and 6,858,229.

In general, these polymers are at least partially soluble in aqueous solutions, such as water, buffered salt solutions, or aqueous alcohol solutions. In some embodiments, the polymers have charged side groups or are monovalent ionic salts thereof. Examples of polymers with acidic side groups that can be reacted with cations are poly(phosphazenes), poly(acrylic acids), poly(methacrylic acids), poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups and sulfonic acid groups.

Examples of polymers with basic side groups that can be reacted with anions are poly(vinyl amines), poly(vinyl pyridine), poly(vinyl imidazole), and some imino substituted polyphosphazenes. The ammonium or quaternary salt of the polymers can also be formed from the backbone nitrogens or pendant imino groups. Examples of basic side groups are amino and imino groups.

A water-soluble gelling agent such as a polysaccharide gum, more preferably a polyanionic polymer like alginate, can be cross-linked with a polycationic polymer (e.g., an amino acid polymer such as polylysine) to form a shell. See e.g., U.S. Pat. Nos. 4,806,355, 4,689,293 and 4,673,566 to Goosen et al.; U.S. Pat. Nos. 4,409,331, 4,407,957, 4,391,909 and 4,352,883 to Lim et al.; U.S. Pat. Nos. 4,749,620 and 4,744,933 to Rha, et al.; and U.S. Pat. No. 5,427,935 to Wang et al. Amino acid polymers that may be used to crosslink hydrogel forming polymers such as alginate include the cationic poly(amino acids) such as polylysine, polyarginine, polyornithine, and copolymers and blends thereof.

### 2. Encapsulated Hydrogels

The expandable hydrogels may be contained within a material, such as a semi-permeable and/or impermeable material. The expandable hydrogels within a semi-permeable and/or impermeable material may be encapsulated in capsules. Alternatively, the hydrogels can be placed directly into a capsule; cast directly onto capsule material during assembly, applied using a thin film coating process such as vacuum deposition or sputter coating, by chemical bonding to the capsule material, or by electrostatic bonding to the capsule material. Suitable capsules include those described in U.S. Publication No. US 2013/0331929.

### 3. Composite Hydrogels

Two or more types of hydrogel may be used together as composite hydrogels. Suitable types of hydrogel include, but are not limited to, poly(acrylic acid), poly(acrylamide), and poly(metacrylic acid), and copolymers and blends of each. The composite hydrogels may be encapsulated within a single capsule. The composite hydrogels may be in a form of strips of the individual types of hydrogel combined into a capsule. This combination of two or more types of gels allows for ionic and non-ionic hydrogels to contribute their swelling characteristics to the swelling of the same capsule. The ionic hydrogels are denser and demonstrate faster swelling for a much lower thickness used, while the non-ionic hydrogels swell slower but more uniformly, and demonstrate long-term stability of the swollen state.

### 4. Calendared Hydrogels

Calendaring hydrogels (mechanically rolling a metal or plastic roller bar over the dried hydrogels with applied pressure) can further reduce the thickness of the dried hydrogels. The thickness can be reduced to about 50% or more of hydrogels' original thickness in a consistent manner. The reduction in thickness allows for crimping a device containing the hydrogel to even lower profiles. The calendering process does not affect the swelling characteristics of the biocompatible hydrogels described herein.

### 5. Hydrogels Containing Protectants

The biocompatible hydrogels may contain protectants against crosslinking or chain scission caused by ionizing radiation. The protectants include, but are not limited to, glycerin, ascorbic acid and trehalose. These compounds “absorb” any free radicals that are produced when the hydrogel is exposed to ionizing radiation, thereby preventing any further crosslinking or other types of radiation damage such as chain scission. Therefore, the hydrogels containing protectants, and any devices that they may be used with, are suitable for sterilization with ionizing radiation.

The added protectants make the biocompatible hydrogels more flexible in dry state, so that they could conform without breaking to a circular prosthesis. The added protectants also make the hydrogels compatible for use with an endoluminal device. The dry hydrogels containing protectants do not break during the crimping and loading process of the device. The added protectants also help to dry the gels to a consistent thickness, making the drying process more robust by protecting against over-drying.

The added protectants do not affect the swelling characteristics of the hydrogels.

### 6. Storage and Sterilization of Hydrogels

The biocompatible hydrogels are formulated to be resistant to changes in organic solvents. The hydrogels are dried down and stored in and/or sterilized with organic solvents without undergoing major changes in thickness of the dried hydrogel. The hydrogels retain their swelling characteristics after storage in and/or sterilization with organic solvents. Most importantly, these hydrogels simplify the storage, sterilization and use of devices containing the hydrogels.

In preferred embodiments, the expandable materials may be stored in storage and/or sterilization solution and do not swell in these solutions during storage/sterilization. A solution for storing and/or sterilizing may be an aqueous solution under conditions suitable for storing natural tissue. Suitable solutions for storage of the expandable materials and devices containing the expandable materials include water miscible solvents, organic and inorganic molecules and salts thereof. Solutions of water miscible organic and inorganic molecules, or their salts, suitable for storage and/or sterilization, include organic compounds glutaraldehyde, formaldehyde, ethanol, propanol, bactericidal or bacteriostatic formulations, fungicide formulations, polyethylene glycol (PEG), polypropylene glycol (PPG), copolymers of PEG and PPG, glycerol, ethanol, isopropanol, acetaldehyde, acetic acid, acetone, acetonitrile, 1,2-butanediol, 1,3-butanediol, 1,4-butanediol, 2-butoxyethanol, butyric acid, diethanolamine, diethylenetriamine, dimethylformamide, dimethoxyethane, dimethyl sulfoxide, 1,4-dioxane, ethylamine, ethylene glycol, formic acid, furfuryl alcohol, glycerol, methanol, methyl diethanolamine, methyl isocyanide, 1-propanol, 1,3-propanediol, 1,5-pentanediol, propanoic acid, propylene glycol, pyridine, tetrahydrofuran, and triethylene glycol; inorganic compounds 1,2-dimethylhydrazine, unsymmetrical dimethylhydrazine, hydrazine, hydrofluoric acid, hydrogen peroxide, nitric acid, sulfuric acid, and other inorganic salts such as sodium chloride, sodium sulfate, and other salts that limit the swelling of the gel due to hydrophobic interactions and/or ionic interactions. The expandable materials do not change their swelling characteristics after storage in or washing with storage/sterilization solutions.

The organic solvents are typically ethanol or isopropanol. In preferred embodiments, the organic solvent is ethanol and ethanol solutions. The ethanol solutions for storage and/or sterilization of the hydrogels, or devices containing the hydrogels, are typically 30-100% ethanol.

For example, a heart valve is typically stored and sterilized in 3.25% glutaraldehyde solution. It is not possible to store and sterilize a heart valve containing an expandable hydrogel without the risk of swelling of the hydrogel within the container. To eliminate this risk, the expandable hydrogel has to be encapsulated within an impermeable barrier (metallic/non-metallic) to shield it from coming in contact with water from the glutaraldehyde solution. The heart valves are also rinsed extensively with PBS before implantation to eliminate residues of glutaraldehyde, which is toxic to live cells. Also, the metallic barrier has to be removed after rinsing and before introduction into the body. These steps can be eliminated with the use of expandable hydrogels compatible with storage and/or sterilization solution in their dried state. If devices contain organic solvent-resistant hydrogels, they may only need rinsing with the storage/sterilization solution prior to implantation.

### 7. Sterilization with Ethylene Oxide

The biocompatible hydrogels are formulated to be resistant to changes after sterilization with ethylene oxide. The hydrogels are dried down and sterilized with ethylene oxide without undergoing changes in thickness of the dried hydrogel. The biocompatible hydrogels retain their swelling characteristics after sterilization with ethylene oxide. These hydrogels, and the devices containing the hydrogels, can be sterilized with ethylene oxide, and then stored in the dry state without affecting the swelling characteristics of the hydrogels.

### 8. Hydrogels Containing Active Agents

It can be advantageous to incorporate one or more therapeutic, prophylactic, diagnostic, or imaging agents (“agent”) into the seal, either by loading the agent(s) into or onto the structural or sealing material. The rate of release of agent may be controlled by a number of methods including varying the following: the ratio of the expandable material to the agent, the molecular weight of the expandable material, the composition of the agent, the composition of the expandable polymer, the coating thickness, the number of coating layers and their relative thicknesses, the agent concentration, and/or physical or chemical binding or linking of the agents to the device or sealing material. Top coats of polymers and other materials, including expandable polymers, may also be applied to control the rate of release.

Exemplary therapeutic agents include, but are not limited to, agents that are anti-inflammatory or immunomodulators, antiproliferative agents, agents which affect migration and extracellular matrix production, agents which affect platelet deposition or formation of thrombis, and agents that promote vascular healing and re-endothelialization. Other active agents may be incorporated. For example, in urological applications, antibiotic agents may be incorporated into the device or device coating for the prevention of infection. In gastroenterological and urological applications, active agents may be incorporated into the device or device coating for the local treatment of carcinoma.

The agent(s) may also include tissue growth promoting materials, drugs, and biologic agents, gene-delivery agents and/or gene-targeting molecules, more specifically, vascular endothelial growth factor, fibroblast growth factor, hepatocyte growth factor, connective tissue growth factor, placenta-derived growth factor, angiopoietin-1 or granulocyte-macrophage colony-stimulating factor.

It may also be advantageous to incorporate in or on the seal a contrast agent, radiopaque markers, or other additives to allow the device to be imaged in vivo for tracking, positioning, and other purposes. Such additives could be added to the expandable composition used to make the seal, or absorbed into, melted onto, or sprayed onto the surface of part or all of the seal. Preferred additives for this purpose include silver, iodine and iodine labeled compounds, barium sulfate, gadolinium oxide, bismuth derivatives, zirconium dioxide, cadmium, tungsten, gold, tantalum, bismuth, platinum, iridium, and rhodium. These additives may be, but are not limited to, micro- or nano-sized particles or nano particles. Radio-opacity may be determined by fluoroscopy or by x-ray analysis.

In some embodiments, one or more low molecular weight active agent such as a therapeutic drug, for example, an anti-inflammatory drug, is covalently attached to the hydrogel forming polymer. In these cases, the low molecular weight drug such as an anti-inflammatory drug is attached to the hydrogel forming polymer via a linking moiety that is designed to be cleaved in vivo. The linking moiety can be designed to be cleaved hydrolytically, enzymatically, or combinations thereof, so as to provide for the sustained release of the low molecular weight drug in vivo. Both the composition of the linking moiety and its point of attachment to the drug are selected so that cleavage of the linking moiety releases either a drug such as an anti-inflammatory agent, or a suitable prodrug thereof. The composition of the linking moiety can also be selected in view of the desired release rate of the drug.

Linking moieties generally include one or more organic functional groups. Examples of suitable organic functional groups include secondary amides (—CONH—), tertiary amides (—CONR—), secondary carbamates (—OCONH—; —NHCOO—), tertiary carbamates (—OCONR—; —NRCOO—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, CHRO₂C—), wherein R is an alkyl group, an aryl group, or a heterocyclic group. In general, the identity of the one or more organic functional groups within the linking moiety can be chosen in view of the desired release rate of the anti-inflammatory agents. In addition, the one or more organic functional groups can be chosen to facilitate the covalent attachment of the anti-inflammatory agents to the hydrogel forming polymer. In preferred embodiments, the linking moiety contains one or more ester linkages which can be cleaved by simple hydrolysis in vivo to release the anti-inflammatory agents.

In certain embodiments, the linking moiety includes one or more of the organic functional groups described above in combination with a spacer group. The spacer group can be composed of any assembly of atoms, including oligomeric and polymeric chains; however, the total number of atoms in the spacer group is preferably between 3 and 200 atoms, more preferably between 3 and 150 atoms, more preferably between 3 and 100 atoms, most preferably between 3 and 50 atoms. Examples of suitable spacer groups include alkyl groups, heteroalkyl groups, alkylaryl groups, oligo- and polyethylene glycol chains, and oligo- and poly(amino acid) chains. Variation of the spacer group provides additional control over the release of the drug in vivo. In embodiments where the linking moiety includes a spacer group, one or more organic functional groups will generally be used to connect the spacer group to both the drug and the hydrogel forming polymer.

In certain embodiments, the one or more drugs are covalently attached to the hydrogel forming polymer via a linking moiety which contains an alkyl group, an ester group, and a hydrazide group.

Reactions and strategies useful for the covalent attachment of drugs to hydrogel forming polymers are known in the art. See, for example, March, “Advanced Organic Chemistry,” 5^{th} Edition, 2001, Wiley-Interscience Publication, New York) and Hermanson, “Bioconjugate Techniques,” 1996, Elsevier Academic Press, U.S.A. Appropriate methods for the covalent attachment of a given drug can be selected in view of the linking moiety desired, as well as the structure of the anti-inflammatory agents and hydrogel forming polymers as a whole as it relates to compatibility of functional groups, protecting group strategies, and the presence of labile bonds.

The seal can further serve as a porous matrix for tissue in-growth and can aid in promoting tissue in-growth, for example, by adding growth factors, etc. This should improve the long-term fixation of the endoluminal prosthesis. For example, the seal can be impregnated with activators (e.g., adhesive activator) that induce rapid activation of the agent (e.g., a tissue adhesive) after the agent has been released from the capsule. In other embodiments, however, the seal can be composed of different materials and/or include different features.

The agent(s) in the capsule can include therapeutic, prophylactic, diagnostic or adhesive materials. Examples include tissue growth promoting materials, and/or gene-delivery or targeting molecules. In another embodiment, the one or more agent may be sheathed for delivery to a target site. Once positioned at the target site, the one or more agent may be unsheathed to enable release to the surrounding environment. This embodiment may have particular application for solid or semi-solid state agents.

Representative adhesives that may be used to aid in securing the seal to the lumen, or to the device to be implanted include one or more of the following cyanoacrylates (including 2-octyl cyanoacrylate, n-butyl cyanoacrylate, iso-butyl-cyanoacrylate and methyl-2- and ethyl-2-cyanoacrylate), albumin based sealants, fibrin glues, resorcinol-formaldehyde glues (e.g., gelatin-resorcinol-formaldehyde), ultraviolet-(UV) light-curable glues (e.g., styrene-derivatized (styrenated) gelatin, poly(ethylene glycol) diacrylate (PEGDA), carboxylated camphorquinone in phosphate-buffered saline (PBS), hydrogel sealants-eosin based primer consisting of a copolymer of polyethylene glycol with acrylate end caps and a sealant consisting of polyethylene glycol and polylactic acid, collagen-based glues and polymethylmethacrylate.

The hydrogel strip can be placed directly into a capsule; cast directly onto capsule material during assembly, applied using a thin film coating process such as vacuum deposition or sputter coating, by chemical bonding to the capsule material, or by electrostatic bonding to the capsule material.

B. Foams and Sponges

Alternatively, a foam generated prior to implantation can also be used as a swellable material to form a seal. For example, a suitable matrix, such as a biocompatible polymer or crosslinkable prepolymer, may be blended with one or more foaming agents. Foaming agents include compounds or mixtures of compounds which generate a gas in response to a stimulus. When dispersed within a matrix and exposed to a stimulus, the foaming agents evolve a gas, causing the matrix to expand as fine gas bubbles become dispersed within the matrix. Examples of suitable foaming agents include compounds which evolve a gas when hydrated with biological fluids, such as mixture of a physiologically acceptable acid (e.g., citric acid or acetic acid) and a physiologically acceptable base (e.g., sodium bicarbonate or calcium carbonate). Other suitable foaming agents are known in the art, and include dry particles containing pressurized gas, such as sugar particles containing carbon dioxide (see, U.S. Pat. No. 3,012,893) or other physiologically acceptable gases (e.g., nitrogen, oxygen, carbon dioxide, argon), and pharmacologically acceptable peroxides.

Other examples include changing the morphology of known hydrogel materials in order to decrease swelling times. Means for changing the morphology include increasing the porosity of the material, for example, by freeze-drying or porogen techniques.

Fast swelling can be achieved by preparing small particles of dried hydrogels. The extremely short diffusion path length of microparticles makes it possible to complete swelling in a matter of minutes. Large dried hydrogels can be made to swell rapidly regardless of their size and shape by creating pores that are interconnected to each other throughout the hydrogel matrix. The interconnected pores allow for fast absorption of water by capillary force. A simple method of making porous hydrogel is to produce gas bubbles during polymerization. Completion of polymerization while the foam is still stable results in formation of superporous hydrogels. Superporous hydrogels can be synthesized in any molds, and thus, three-dimensional structure of any shape can be easily made. The size of pores produced by the gas blowing (or foaming) method is in the order of 100 μm.

If any portion of a superporous hydrogel is in contact with water or an aqueous medium, water is absorbed immediately through the open channels to fill the whole space. This process makes the dried superporous hydrogels swell very quickly.

Expandable sponges or foams can also be used for sealing of surgical implantations. These sponges or foams and be cut into a strips or annular shapes and either dried down or dehydrated by other means and then be allowed to rapidly re-hydrate once the device is in place. Alternatively, such materials can be hydrated and then squeezed to reduce their volume to allow these to be attached to the surgical implement and then allowed to expand to form a seal once the surgical implement is in place. Such swelling would be nearly instant. One further benefit of sealing material in the form of sponges or foams is that their expansion can be reversible so that they can easier be retracted from their implanted position back into the delivery catheter and thereby enable complete re-positioning of the device multiple times and/or complete retrievability of the device. Such sponges and foams can be made from a range of materials including, but not limited to, synthetic polymers, natural polymers or mixtures thereof. Such materials can be formed by including pore forming substances such as gas or immiscible solvents in the monomer/polymer mix prior to polymerization and/or cross-linking. By using the appropriate monomers and/or polymeric cross-linkers such sponges/foams can be made to withstand cyclic stress; such materials could also further be reinforced with compatible fibers or whiskers to increase strength and reduce the probability for breakage.

In some embodiments, these sponges or foams can be chemically attached to a base membrane or mesh used to encapsulate the sponge/foam before being fitted to the surgical device. This could be done by attaching either allylic or acrylic groups to the base substrate, either as small molecules or as long chain tentacles anchoring the expandable to the substrate preventing release of smaller particles in case of fracture.

Foams may be designed to expand without the need for the semi-permeable membrane.

### III. Endovascular Devices

Endovascular devices suitable for use with the seals described herein include, but are not limited to, embolization devices, devices for Abdominal Aortic Aneurysm (AAA) repair, and Thoracic Aortic Aneurysm (TAA) repair devices.

Suitable devices for AAA repair, including complex AAA, are summarized in Jackson et al., Seminars in Interventional Radiology, 26:39-43 (2009) and Singh et al., Endovasular Today, February 2013:63-66 (2013), and include, but are not limited to, single channel, bifurcated, branched, fenestrated stent grafts, such as Zenith Flex® by Cook Medical © (Bloomington, Ind.), AneuRx® by Medtronic (Minneapolis, Minn.), Talent® by Medtronic (Minneapolis, Minn.), Powerlink® by Endologix (Irvine, Calif.), and Excluder® by Gore Medical (Flagstaff, Ariz.).

Thoracic Aortic Aneurysm (TAA) or ulcers of the descending thoracic aorta having vascular morphology suitable for endovascular repair may be repaired or treated with the TAA devices and the seal described herein.

The seals described herein are also compatible for use with the chimney endovascular aneurysm repair (ChEVAR) devices. The chimney technique in endovascular aortic aneurysm repair (Ch-EVAR) involves placement of a stent or stent-graft parallel to the main aortic stent-graft to extend the proximal or distal sealing zone while maintaining side branch patency. Ch-EVAR can facilitate endovascular repair of juxtarenal and aortic arch pathology using available standard aortic stent-grafts, therefore, eliminating the manufacturing delays required for customized fenestrated and branched stent-grafts (Patel et al., Cardio Vascular and Interventional Radiology, 36(6):1443-1451 (2013)).

Examples of other endovascular devices suitable for use with the seal are described in U.S. Publication No. US 2013/0331929, US 2013/0190857, US 2013/0197622, and US 2011/0282426.

### IV. Kits

Also provided are kits containing at least one seal and at least one endovascular device. The kits may provide a plurality of seals for use with various endoluminal devices, of various diameters and surgical methods of implantation. The kits may also contain instruments for implanting the device with the seal into the body lumen.

Suitable devices and seals are described above. For example, kits may contain bifurcated stent grafts and seals as separate devices for the assembly into sealing devices by the user. Alternatively, the kits can provide the stent grafts with seals attached and ready for use.

In other embodiments, the kits may contain ChEVAR devices and seals as separate devices for assembly by the user. In other embodiments, the kits may provide a plurality of chimneys for the ChEVAR device separately from or already attached to the seal, and the stent grafts with or without the seal. The assembly of the ChEVAR devices into a sealing device can be performed by the user prior to use. Kits may also provide chimneys and stent grafts with the seals already assembled and ready to use.

### V. Methods of Use

Generally, the expandable materials described herein may be used alone or in combination with other materials. When used in combination with other materials, the expandable materials may be incorporated into seals, and serve as the expandable materials of seals to seal devices to tissues. Alternatively, the expandable materials may be used as fillers for aneurysm sac filling, or as sealers for sealing the aneurysm neck from the surrounding blood flow.

In some embodiments, these expandable materials can be spray dried onto, or covalently attached to, a base membrane or mesh used to encapsulate the gel before being fitted to a surgical device. The gels can be covalently attached by introducing one or more functional groups that can form covalent bonds to one or more functional groups on the base membrane or mesh. Suitable functional groups include, but are not limited to, allylic, vinyl or acrylic groups. The functional groups can be introduced directly onto the gel and/or membrane or mesh or as part of a longer/larger chemical moiety. “Allyl”, as used herein, refers to a group having the structural formula H2C═CH—CH2R, where R is the point of connection to the rest of the molecule, i.e., hydrogel and/or base membrane or mesh. “Acrylic”, as used herein, refers to a group having the structure H₂C═CH—C(═O)—. The preferred IUPAC name for the group is prop-2-enoyl, and it is also (less correctly) known as acrylyl or simply acryl. Compounds containing an acryloyl group can be referred to as “acrylic compounds”. “Vinyl”, as used herein, refers to a group containing the moiety —CH═CH₂, which is a derivatives of ethene, CH₂═CH₂, with one hydrogen atom replaced with some other group or bond, such as a bond to the base substrate or membrane. Vinyl groups can be introduced directly onto the hydrogel and/or base membrane or mesh or can be part of a longer/larger chain.

The long chain hydrophilic crosslinking agents described above have at least two and preferably more than two reactive functional groups (e.g., allyl, acrylic, vinyl, etc.) capable of participating in a free radical polymerization reaction or additional reaction, such as Michael addition, and where at least part of the molecule is attached to a substrate, anchoring the gel to the substrate to prevent release of smaller gel particles in case of gel fracture.

Long-chain cross-linkers and/or the chemical attachment of the gels to a porous substrate result in gels that are more capable of withstanding cyclic loads. These seals containing gels can be made in any shape, including annular or strip shape. The principle behind these cross-linkers is that rather than having a short cross-linker with only two polymerizable groups, the crosslinking agents described herein includes long chain hydrophilic polymer (such as PVA, PEG, PVAc, natural polysaccharides such as dextran, HA, agarose, and starch) with multiple polymerizable/reactive groups. The long chain crosslinking agents result in a hydrogel which is less susceptible to “fragmenting” which is important as it minimizes any risk of small gel particles breaking off and embolizing to the brain. The long chain crosslinking agents also result in increased integrity of the hydrogel, making it more pliable and thereby increasingly resilient under cyclic loads, an important factor for long-term durability of the hydrogel. These stronger gels are synthesized using long chain cross-linkers, typically molecules with more than 20 carbon atoms and/or a molecular weight greater than 400 Da, more preferably more than 40 carbon atoms and/or a molecular weight greater than 800 Da, that will act as molecular reinforcement molecules, creating a more resilient and longer lasting gel while maintaining excellent swelling properties. The swelling force of these gels can also be adjusted to not exert more radial force than necessary, typically around 0.00005 N/mm² to 0.025 N/mm², preferably 0.002 N/mm² to 0.012 N/mm².

While these gels are very firm, they at the same time possess very good swelling characteristics. Very strong gels do not swell as much and/or as rapidly. As used herein, very strong refers generally to hydrogels having a strength greater than about 0.00005 N/mm² to 0.025 N/mm². Desired swelling ratios are 20× or greater, with an ideal range of 20×-60×. The greater the swelling ratio, the smaller the introduction profile of the device, allowing treatment of a greater number of patients who have smaller access vessels (femoral arteries, radial arteries, etc.).

In all embodiments, it is absolutely critical that the hydrogel/expandable material operates under sufficient low pressure so that it does not push the stent away from the wall or alter the device configuration.

In summary, the expandable material is contained within a material such as a semi-permeable or impermeable material so that it is retained at the site where it is needed to seal a leak. The material is selected based on the means for activation. If the material is expanded by mechanical shear or exposure to a foaming agent, these materials are provided internally within the seal, allowing an external activating agent such as an activation wire to disrupt the means for isolating the activation agent from the expandable material.

If the material is activated by contact with fluid, no additional means for isolation are required if the device is stored dry prior to use, since it will activate in situ when exposed to body fluids. If the material is stored wet prior to use, a second impermeable membrane may be used to keep the expandable material dry prior to activation. This will typically include a rupture site which is opened at the time of implantation to allow biological fluid to reach the expandable material through the semi-permeable material (i.e., where semi-permeable refers to a material retaining the expandable material but allowing fluid to pass). Alternatively the impermeable material may not include a rupture site but simply be removed after the device is removed from storage and washed with saline, prior to loading into the catheter, so that once the device is deployed, in situ liquid will cause the hydrogel to swell.

The properties of the different materials complement each other. For example, in the time immediately after valve deployment it is important that the material swells quickly to seal perivalvular leaks as soon as possible. Mechanical strength may be compromised in the short term to enable fast swelling. In the long term, however, it is paramount that the seal has high mechanical strength. In some embodiments, the mechanical strength of the hydrogel(s) is from about 0.00005 N/mm² to about 0.025 N/mm², preferably from about 0.002 N/mm² to about 0.012 N/mm². The mechanical strength should be high enough to allow swelling and thereby “actively” conform to the gaps leading to leakage but not high enough to disturb the physical or functional integrity of the prosthesis or implant or to push the prosthesis or implant away from the wall. Another important consideration is that the mechanical strength should not be so high as to exert excess pressure on the anatomy, particularly around the Left Bundle Branch (LBB), which is responsible for the cardiac conduction. If excess pressure is exerted a cardiac conduction abnormality known as the Left Bundle Branch Block (LBBB) may occur. Typically, it is taken into consideration that the outward pressure exerted on the anatomy by the swelling of the hydrogel is less than that exerted by the prosthesis or implant.

A degradable material, which may be a hydrogel, that swells quickly, may be used in conjunction with a nondegradable material, which may be a hydrogel that swells slower but has higher mechanical strength. In the short term, the degradable material capable of rapid swelling will quickly seal the perivalvular leak. Over time, this material degrades and will be replaced by the material exhibiting slower swelling and higher mechanical strength. Eventually, the seal will be composed of the slower swelling nondegradable material. It is also possible to use only one material in the seal, but in two or more different forms. For example, two different crystal sizes of hydrogels may be used in the seal, because different particle sizes of hydrogel may exhibit different properties.

A. Use as a Seal

The seal includes a flexible component, such as an expandable material, that is configured to conform to irregularities between an endoluminal prosthesis and a vessel wall. The seal includes a generally ring-like structure having a first or inner surface and a second or outer surface. It contains a material that swells upon contact with a fluid or upon activation of a foam, following placement, to inflate and conform the seal around the device. The swellable material within the seal may be enclosed in a capsule.

The seal can be provided in a variety of shapes, depending on the device it is to be used with. A “D” shape is the preferred embodiment, with the flat portion being attached to a support structure (support member) and/or device to be implanted.

The seal can be composed of a permeable, semi-permeable, or impermeable material. It may be biostable or biodegradable. For example, the seal may be composed of natural or synthetic polymers such as polyacrylic acid, polyacrylamide, polyether or polyester polyurethanes, polyvinyl alcohol (PVA), silicone, cellulose of low to high density, having small, large, or twin pore sizes, and having the following features: closed or open cell, flexible or semi-rigid, plain, melamine, or post-treated impregnated foams. Additional materials for the seal can include polyvinyl acetal sponge, silicone sponge rubber, closed cell silicone sponges, silicone foam, and fluorosilicone sponge. Specially designed structures using vascular graft materials including polytetrafluoroethylene (PTFE), polyethylterephthalate (PET), polyether ether ketone (PEEK), polyurethane (PU), woven yarns of nylon, polypropylene (PP), collagen or protein based matrix may also be used. PEEK is the preferred material at this time since the strength is high so that there will be no damage leading to failure when the TAV device is expanded against sharp/calcified nodules and at the same time a relatively thin sheet of material can be used, helping maintain a lower profile. In preferred embodiments, the seal includes polyacrylamide and/or polyacrylic acid.

The seal material may be used independently or in combination with a mesh made from other types of polymers, titanium, surgical steel or shape memory alloys.

### 1. Capsules

In some embodiments, the seal may include one or more capsules. The capsule may be segmented to include one or more compartments. The compartments may be relatively closely spaced. Further, the distance between adjacent compartments may vary. The segmented capsule of this embodiment may not extend completely around the endoluminal prosthesis.

The capsule may include an outer wall to hold the agent therein. The outer wall may be made of a suitably flexible and biocompatible material. Alternatively, the capsule may include a more rigid structure having a pre-designed failure mechanism to allow the release of agent therefrom. Examples of suitable materials include, but are not limited to, low density polyethylene, high density polyethylene, polypropylene, polyurethane, polytetrafluoroethylene, silicone, or fluorosilicone. Other fluoropolymers that may be used for the construction of the capsule include: polytetrafluoroethylene, perfluoroalkoxy polymer resin, fluorinated ethylene-propylene, polyethylenetetrafluoroethylene, polyvinylfluoride, ethylenechlorotrifluoroethylene, polyvinylidene fluoride, polylychlorotrifluoroethylene, perfluoropolyether, fluorinated ethylene propylene, terpolymer of tetrafluoroethylene, hexafluoropropylene and vinylidene fluoride), polysulphone and polyether ether ketone (PEEK). It may also include non-polymeric materials such as glass, bioglass, ceramic, platinum and titanium. It may further include biologically based materials such as crosslinked collagen or alginates. It will be appreciated that the foregoing list is provided merely as an example of suitable materials and is not an exhaustive list. The capsule may be composed of a material or combination of materials different from those provided above.

Typically, the capsule is configured to rupture to release one or more agents at a predetermined range of pressures. The range of rupture pressures includes between 5 and 250 psi, between 5 and 125 psi, between 10 and 75 psi, or at approximately 50 psi.

A variety of different techniques or processes can be used to form pressure activated capsules or compartments. In one embodiment, for example, a process for forming a pressure activated capsule includes pre-stressing the capsule during formation. The pre-stressed material will have a limited capacity to stretch when subjected to external pressure, and will fail when reaching critical stress on the stress-strain curve. The first stage of this method includes selecting a biocompatible capsule material that is also compatible with its contents (e.g., the agent which can include adhesive material or a wide variety of other types of materials). The capsule material should also have a tensile strength suitable for the particular application in which the capsule will be used.

The next stage of this method includes forming an undersized capsule. The undersized capsule is essentially shaped as an extruded, elongated tube (e.g., a “sausage”) with one end of the tube sealed (e.g., by dipping, dip molding, vacuum forming blow molding, etc.). The process continues by expanding the capsule to its final shape. The capsule can be expanded, for example, by stretching (e.g., either hot or cold) using appropriate tooling so that the capsule material is pre-stressed to within a stress level, and whereby the clinically relevant balloon inflation pressure will exceed the failure stress of the capsule material. The method can further include filling the capsule with the desired contents while the capsule is under pressure so as to achieve pre-stressing in a single step. After filling the capsule, the capsule can be sealed (e.g., using a heat welding process, laser welding process, solvent welding process, etc.).

In another embodiment, a capsule can be formed by forming an air pillow or bubble wrap-type capsule using a vacuum form process or other suitable technique. The next stage of this process includes perforating a film at the base of the capsule assembly and filling the individual capsules with the desired contents under an inert atmosphere. After filling the capsules, the puncture hole can be resealed by application of another film over the puncture hole and localized application of heat and/or solvent. Other methods can be used to seal the puncture hole. In several embodiments, the capsule can be configured such that the puncture hole re-ruptures at the same pressure as the capsule itself so that there is some agent (e.g., adhesive material within the capsule) flowing onto the corresponding portion of the endoluminal prosthesis.

One or more failure points can be created within a capsule. This process can include creating a capsule shaped as an extruded, elongated tube with one end of the tube sealed (e.g., by dipping, dip molding, vacuum forming blow molding, etc.). The capsule can be composed of a polymer material (e.g., polyethylene, polypropylene, polyolefin, polytetrafluoroethylenes, and silicone rubber) or another suitable material. At one or more predetermined locations along the elongated tube, the process can include creating areas of substantially reduced thickness. These areas can be formed, for example, using a tool (e.g., a core pin with a razor blade finish along the length of the capsule), laser ablation, creating partially penetrating holes, creating an axial adhesive joint (e.g., tube from a sheet) that is weaker than the substrate, or other suitable techniques. The method next includes filing the capsule with the desired contents at a pressure below that required to rupture the thinned or weakened areas. After filling the capsule, the open end of the capsule can be sealed using one of the welding processes described above or other suitable processes.

In yet another particular embodiment, one or more stress points can be created within a capsule. This method can include forming a capsule and filling the capsule with the desired contents using any of the techniques described above. After forming the capsule and with the capsule in an undeployed configuration, the process can further include wrapping a suture (e.g., a nitinol wire) about the capsule at a predetermined pitch and tension. When the capsule is moved from the undeployed state to a deployed configuration and takes on a curved or circumferential shape, the suture compresses the capsule at the predetermined points. Stress points are created in the capsule walls at these points because of the increased pressure at such points.

In another embodiment the device may include one or more pressure points on the supporting member such as spikes or other raised areas which cause the penetration of the capsule once a predetermined pressure is applied thereto.

Still yet another particular embodiment for forming a pressure activated capsule or compartment includes creating a double walled capsule in which an inner compartment of the capsule is sealed and separated from an outer compartment of the capsule that contains the adhesive or other desired agent. The inner compartment can be composed of a compliant or flexible material, and the outer compartment can be composed of a substantially less compliant material. The outer compartment may or may not have failure points. The inner compartment is in fluid communication via a one way valve with a low compliance reservoir. The reservoir is configured to be pressurized by inflation of an expandable member or balloon to a high pressure, thereby allowing the valve to open and pressurize and expand the inner compartment. This process in turn pressurizes the outer compartment (that contains the adhesive) until the outer compartment ruptures. One advantage of this particular embodiment is that it can increase the pressure within the capsule to a value higher than otherwise possible with an external expandable member or balloon alone.

In a still further embodiment, the capsule has an inner compartment made from a relatively rigid material or mesh and an outer compartment made from a relatively flexible material. In this embodiment, the inner compartment acts as a reservoir, containing the agent and is designed to break or rupture at a predetermined pressure. The outer compartment may also have a failure pressure point to allow release of the agent. The rigidity of the inner compartment may provide a longer-term stability and shelf life of the encapsulated agent. The application of rupture pressure may be carried out either locally or remotely, e.g. via a tube directly connected to the capsule that is connected to an external source at the delivery device entry site (e.g. femoral artery).

### 2. Expandable Capsule

In one embodiment, a seal entirely surrounds the capsule such that the capsule is “suspended” within the seal. In one specific embodiment, for example, the seal can include a porous material configured to prevent any embolization (distal or proximal) of released agent(s) from the capsule. The seal may have a graded degree of relative porosity from relatively porous to relatively non-porous. Preferred porosity size is from five to seventy microns, more preferably about 35 microns so that the fluid can rapidly access the swellable material.

In the preferred embodiment, the capsule is a single annular compartment within the seal, and extends completely around the periphery of the endoluminal prosthesis. In other embodiments, however, the capsule may include one or more additional compartments or sections, and may not extend completely around the endoluminal prosthesis. Moreover, the capsule may or may not be contained within the seal, and can be positioned at a different location on the apparatus relative to the seal. In addition, the capsule can have a variety of different shapes and/or sizes depending upon the particular application, the agent(s), the configuration of the endoluminal prosthesis, and a number of other factors.

### 3. Permeable and Impermeable Membranes

The capsule can have both inner and outer membranes, either or both of which may be permeable, at least one of which has to be permeable.

In a preferred embodiment, the seal includes a permeable membrane. In another embodiment, the seal also includes an impermeable membrane. In this embodiment, the permeable membrane is an inner membrane, and the impermeable membrane is an outer membrane. In all embodiments, an expandable material such as a foam or hydrogel is placed within the permeable membrane. In some embodiments, the membrane is semi-permeable and encapsulates the expandable material. This membrane is semi-permeable and allows fluid ingress but not egress of entrapped hydrogel or foam. The outer membrane is impermeable except at an optional pre-determined rupture point. The outer membrane is designed to be impermeable to fluid during storage and transport and during any pre-procedural preparations e.g. rinsing or washing of the device, to protect the polymer from premature swelling. The outer membrane is also designed to be strong and puncture resistant so that it does not tear or is punctured or pierced by the sharp edges of the native calcification even when subject to pressures up to 14 atm. This prevents the rupture of the permeable membrane, mitigating any risk of embolization of the expandable material or hydrogel. The rupture point allows fluid such as blood to penetrate into the expandable seal only when the seal is expanded in place, thereby preventing leaks.

Permeable membranes may be made from a variety of polymer or organic materials, including polyimides, phospholipid bilayer, thin film composite membranes (TFC or TFM), cellulose ester membranes (CEM), charge mosaic membranes (CMM), bipolar membranes (BPM), and anion exchange membranes (AEM).

A preferred pore size range for allowing fluid in but not hydrogel to escape is from five to seventy microns, more preferably about 35 to seventy microns, most preferably about 35 microns, so that the fluid can rapidly access the swellable material.

The permeable membrane may be formed only of permeable material, or may have one or more areas that are impermeable. This may be used to insure that swelling does not disrupt the shape of the seal in an undesirable area, such as on the interior of the device where it abuts the implant or prosthesis, or where it contacts the device support members.

In some embodiments, the second impermeable membrane is applied with plasma vapor deposition, vacuum deposition, co-extrusion, or press lamination.

B. Use with Endoluminal Devices

Endoluminal devices, containing a prosthesis and a seal, are advanced through a body lumen in a first undeployed and reduced profile configuration. When positioned in situ, the sealing device expands from its reduced radial profile configuration to a second configuration with an increased radial profile. In situ, and in its second configuration, the sealing device is configured to be positioned between the prosthesis and the wall of the body lumen. In one embodiment, when the endoluminal prosthesis is at the desired location in the body lumen, it is typically deployed from an introducer catheter whereupon it may move to an expanded radial configuration by a number of mechanisms. In some embodiments, the prosthesis may be spring expandable. Alternatively, a balloon or expandable member can be inflated within the lumen of the prosthesis to cause it to move to an expanded radial configuration within the vessel. This radial expansion, in turn, presses the sealing device against a wall of the body lumen. One of the advantages of the seal is that it only fills the gaps, and does not impact the placement and integrity—both physical and functional, of the prosthetic or the implant.

In one embodiment, the sealing device is configured to fully seal a proximal, central and/or distal end of the endoluminal prosthesis for endovascular aneurysm repair (EVAR) to prevent endoleaks and prevent subsequent migration and/or dislodgement of the prosthesis.

In another embodiment, the sealing device is configured to fully seal a transcatheter aortic valve.

The seal may be configured such that it moves independently of the endoluminal prosthesis. Alternatively, the seal may be connected to the prosthesis for delivery to a target site. The seal may be connected to the prosthesis by any number of means including suturing, crimping, elastic members, magnetic or adhesive connection.

In one embodiment, the sealing means is positioned posterior to the prosthetic implant, and is expanded and pulled up into a position adjacent to the implant at the time of sealing. This is achieved using sutures or elastic means to pull the seal up and around the implant at the time of placement, having a seal that expands up around implant, and/or crimping the seal so that it moves up around implant when implant comes out of introducer sheath. This is extremely important with large diameter implants such as aortic valves, which are already at risk of damage to the blood vessel walls during transport.

A key feature of the latter embodiment of the seal technology is that it enables preservation of the crimped profile of the endoluminal prosthesis. The seal technology is positioned distal or proximal to the prosthesis. In one aspect of this technology, the seal is aligned with the prosthesis by expansion of the seal. In another aspect, the seal zone of the prosthesis is aligned with the seal zone prior to expansion of the prosthesis. In additional embodiments, the seal is positioned between the device skeleton and the device, or on the exterior of the skeleton.

In a further embodiment, the seal may further include one or more engagement members. The one or more engagement members may include staples, hooks or other means to engage with a vessel wall, thus securing the device thereto.

Other suitable devices for use with the seal described herein include devices described in U.S. Publication Nos. US 2013/0331929, US 2013/0190857, US 2013/0197622, and US 2011/0282426. It is to be understood by those skilled in the art, that the use of the seal described herein is not limited to the disclosed devices, and any other device, benefiting from the use with the described seal, may be used.

C. Use of Hydrogels as Fillers or as Sealers

1. Filling Aneurysm Sac

The goal of embolization is to selectively obliterate an abnormal vascular structure, while preserving blood supply to surrounding normal tissues. When embolizing aneurysms, the hydrogel is positioned within the aneurysm sac and fully seals the neck of the aneurysm from the blood flow of the vessel. The hydrogel may be positioned fully within the aneurysm sac, so that it contacts the walls of the sac, fills the sac completely or only 90%, 80%, 70%, 60%, 50%, 40%, 30%, 20%, or 10% of the sac cavity, while fully sealing the aneurysm neck from the surrounding blood supply. Alternatively, the gel can be hydrated by an aqueous liquid, e.g. sterile distilled water, sterile saline, supplied from an external source. The swelling of the gel can be regulated by monitoring the amount of aqueous liquid supplied externally.

The substantially dry hydrogel materials may be introduced with a catheter under radiographic guidance to the aneurysm. Upon delivery, the hydrogel, which may be in a shape of in rod, pellet, fiber, rolled up film or other physical form, may rehydrate and occlude the vascular flow by mechanical obstruction.

The hydrogel material may be free of membranes, or be contained within, or encapsulated by, porous membranes. The hydrogel material may include one or more types of gels, such as composite gels, copolymer gels, or combinations thereof. The hydrogel material with or without membranes, may be encapsulated. The encapsulated hydrogels, or hydrogels in free form, may include prophylactic, therapeutic, diagnostic, adhesive, or imaging agents. The agents may be loaded in capsules. The agents may be incorporated in the polymer network of the hydrogels. The agents may be released upon delivery of the gel material via controlled release from the capsules. The agents may be released via diffusion from the polymer network of hydrogels. The agents may be released sequentially. The adhesive may be released to seal the points of contact of the gel material with the vessel walls.

2. Sealing Endoluminal Devices to Vessel Walls

The endoluminal devices with hydrogel seals can be utilized for sealing in a variety of tissue lumens, including cardiac chambers, cardiac appendages, cardiac walls, cardiac valves, arteries, veins, nasal passages, sinuses, trachea, bronchi, oral cavity, esophagus, small intestine, large intestine, anus, ureters, bladder, urethra, vagina, uterus, fallopian tubes, biliary tract or auditory canals. In operation, the endoluminal prosthesis is positioned intravascularly within a patient so that the prosthesis is at a desired location along a vessel wall. A balloon or other expandable member is then expanded radially from within the endoluminal prosthesis to press or force the apparatus against the vessel wall. As the balloon expands, the activation wire is triggered, rupturing the capsule and causing the seal to swell, and in some embodiment, releasing agents. In one embodiment, the agent includes an adhesive material and when the capsule ruptures, the adhesive material flows through the pores of the seal. As discussed above, the seal can control the flow of the adhesive to prevent embolization of the adhesive material.

In specific embodiments, the device may be used to seal a graft or stent within an aorta of a patient. In a further embodiment, the device may be used to seal an atrial appendage. In this embodiment, the device may deliver an agent to effect the seal of a prosthetic component across the opening to the atrial appendage.

In a further embodiment, the device may be used to seal a dissection in a vessel. In this embodiment, the support member is positioned adjacent the opening of the false lumen and an intraluminal stent subsequently delivered thereto. Upon radial expansion of the stent, the support member is caused to release adhesive therefrom to seal the tissue creating the false lumen against the true vessel wall.

In a further embodiment, the device is used to seal one or more emphysematous vessels.

In a still further embodiment, the device may be used to seal an artificial valve within a vessel or tissue structure such as the heart. An example includes the sealing of an artificial heart valve such as a TAV.

The device with seal is inserted in a manner typical for the particular device. After reaching the implantation site, the seal is ruptured and the seal expands to seal the site. The guidewire and insertion catheter are then withdrawn and the insertion site closed.

The seal may be sterile packaged for distribution and use. In the alternative, it may be packaged as part of, or in a kit with, the device it is designed to seal, such as a TAV or stent.

3. Sealing Endoleaks or Preventing Paravalvular Leaks

In one embodiment, the hydrogel seal and the endoluminal device may be used to seal endoleaks or to prevent paravalvular leaks. When used for sealing endoleaks, the device is positioned within the lumen of a vessel with an aneurysm, oversizing the aneurysm. The seal of the device is allowed to expand and contact the walls of the vessel. In one embodiment, the expanded seal contacts the vessel walls at regions above and/or below the aneurysm sac. In another embodiment, the expanded seal contacts the walls of the aneurysm sac itself, filling the sac. In another embodiment, the seal contacts the walls of the aneurysm sac partially filling the sac. The expanded seal may fill the aneurysm sac completely and tightly contact the sac wall with an endoleak sealing the endoleak. In another embodiment, the expanded seal may partially fill the aneurysm sac so as to prevent blood from entering the sac and stopping the endoleak.

The seal operates under sufficient low pressure so that it does not push the stent away from the vessel wall or alter the device configuration while securely sealed to the vessel wall. The seal also actively conforms to vascular anatomy at the site of implantation and to any alterations in lumen size following implantation. These characteristics allow the seal to preventing paravalvular leaks.

Once in position within the vessel lumen, the seal seals the device to vessel walls and/or aneurysm sac, and allows the blood to pass through the lumen of the device. This prevents the blood from contacting the walls of the aneurysm sac, further reducing the possibility of endoleaks.

### VI. Examples

### A. Materials and Methods

Hydrogels formed of acrylamide monomers crosslinked with N,N′-methylenebisacrylamide, poly(ethylene glycol) diacrylate (PEGDA) or poly(ethylene glycol) diacrylamide (PEGDAA), were used. The monomer and the crosslinker were mixed first followed by addition of initiators ammonium persulfate (APS) and N,N,N′,N′-tetramethylethylenediamine (TEMED) The concentration of monomer is typically between 7% and 15%. The various formulations of acrylamide monomers and the crosslinkers used are presented in Table 1.

The gel was then cast between two glass plates separated by a spacer of about 750 p.m. The gel was removed from the glass plates and washed in an appropriate washing solution, typically with three changes of the solution. The composition of the washing solution was typically 20-50% ethanol and 0.1%-1% glycerine. The resulting concentration of glycerine in the dried gel was estimated to be around 1%-10%. Glycerin was added to enhance the flexibility of gels and to function as a free-radical scavenger during sterilization with ionizing radiation.

The washing solution also contained salts to neutralize unreacted charges. The salt was typically sodium chloride and the concentration was between 0.1-3 M in the solution.

After washing, the gel was placed between two pieces of mesh and the gel-mesh assembly was dried in a slab gel dryer under vacuum. After drying, the gel was detached from mesh and pressed by rolling a polymer or metal roller over the gel while applying pressure (calendaring). The resulting hydrogels were in a sheet form and had a thickness as low as 10-20 μm or 50-70 μm, when in dry state.

For composite gels, acrylamide gels were combined with acrylic acid gels and crosslinked with N,N methylenebisacrylamide. Acrylic acid monomers were crosslinked with poly(ethylene glycol) diacrylate (PEGDA), at the monomer concentration of 15%.

The gels were cut into pieces of typically 1 cm by 1 cm squares, weighed and then submerged into PBS, serum or other test liquid and allowed to swell. At given time points, the gel pieces were separated from the liquid with the help of a sieve. Excess liquid was removed by gentle blotting and the gel was weighed. Following weighing, the gel was re-submerged in the liquid for re-measuring at the next time point. At least three gels per condition were tested. The data are a mean of the individual measurements per condition.

To test for the swelling characteristics of the gel, the weight of a gel piece in dry state, and at various time points following placement in liquid, was compared. The ratio of the weight at a particular time point over the weight in dry state, multiplied by 100%, was used to represent the swelling ratio of the gels in percentages (%).

Alternatively, the pieces were placed in ethanol solution and then tested for the swelling characteristics. The pieces were also tested for the swelling characteristics following sterilization with ethylene oxide and E-beam.

B. Results

Results of the swelling characteristics obtained from the various acrylamide hydrogels is presented in Table 1.

TABLE 1
Ratios of monomers and crosslinkers used for different formulations of
gels, and their corresponding swelling ratios in PBS and bovine serum.

| | | | | | Swelling |
|---|---|---|---|---|---|
| | | | | Swelling | ratio (%) |
| Monomer | Crosslinker | | Ratio | ratio (%) | in bovine |
| (M) | (L) | T-C¹ | M:L² | in PBS | serum |
| Acrylamide | N,N′-Methylene- | T10-C0.06 | 1667:1 | ~4200 | ~3000 |
| | bisacrylamide | | | | |
| Acrylamide | N,N′-Methylene- | T10-C0.07 | 1429:1 | ~3800 | ~3000 |
| | bisacrylamide | | | | |
| Acrylamide | N,N′-Methylene- | T10-C0.08 | 1250:1 | ~3700 | ~3100 |
| | bisacrylamide | | | | |
| Acrylamide | N,N′-Methylene- | T9.4-C0.06 | 1667:1 | ~4800 | ~3400 |
| | bisacrylamide | | | | |
| Acrylamide | Poly(ethylene | T7-C2 | 50:1 | ~5400 | ~3800 |
| | glycol)diacrylamide | | | | |
| Acrylamide | Poly(ethylene | T7-C3 | 33:1 | ~3800 | ~3000 |
| | glycol)diacrylamide | | | | |
| Acrylamide | Poly(ethylene | T8-C2 | 50:1 | ~4000 | ~3000 |
| | glycol)diacrylamide | | | | |
| Acrylamide | Poly(ethylene | T9-C2 | 50:1 | ~3900 | ~2900 |
| | glycol)diacrylamide | | | | |

¹T: (concentration of monomer) × 100; C: (crosslinker/monomer (w/w)) × 100
²Ratio of monomer (M) to crosslinker (L) in w:w

### Example 1

### Rapid Swelling of Acrylamide Gels in Serum

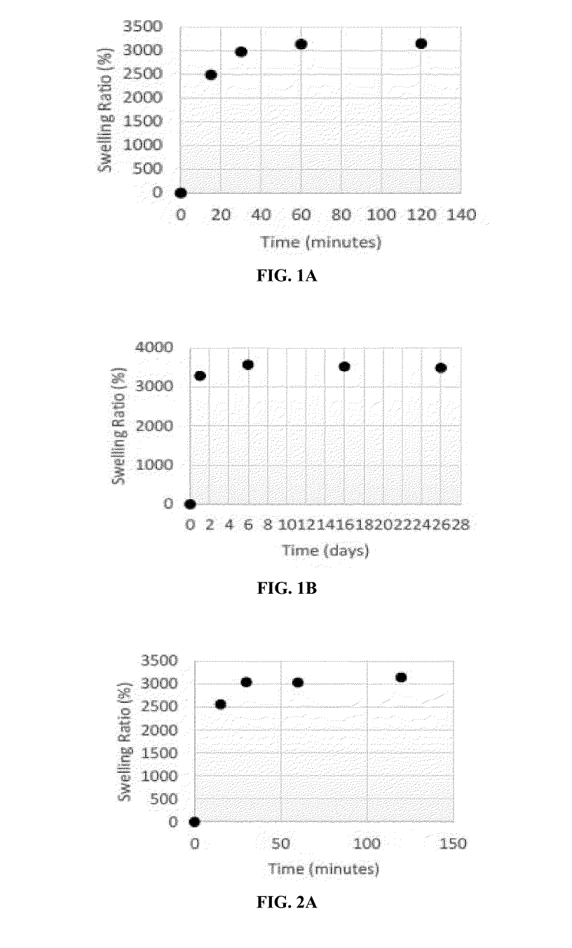

Acrylamide gels (T10-00.06) demonstrated a rapid swelling with a swelling ratio of 3000% when placed in bovine serum. The gels swelled rapidly within the initial 15 min, and then reached a maximum swelling ratio of 3500% when stored in bovine serum long term (FIGS. 1A and 1B). The acrylamide gels retained this swollen state over long-term storage in serum, demonstrating long-term stability of the gels in bovine serum.

The rapid swelling and the stability of the gels are provided by the ratio of monomer to crosslinker (w:w), as presented in Table 1. The concentration of the crosslinker with respect to the monomers is low enough to allow for formation of pores with long polymer chains. The pores with long polymer chains maintain their swelling capacity after drying and rehydration of the gels. These pores also retain their size following rehydration and provide for the long-term stability of the gels.

### Example 2

### Protectants do not Affect the Swelling or Stability of Acrylamide Gels

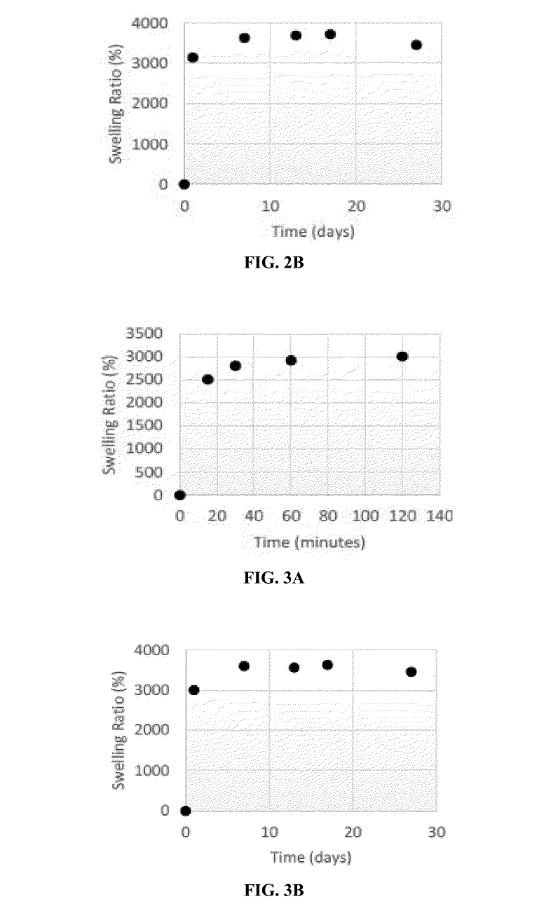

Acrylamide gels containing the protectant glycerin also demonstrated a rapid swelling and reached a swelling ratio of 3000% within the initial 15 min of swelling. Pure acrylamide gels containing 0.1% to 1% glycerin swelled to the swelling ratio of over 3500% when stored long-term in bovine serum (FIGS. 2A-3B). The swelling ratio of gels containing glycerin also remained stable over time, demonstrating long-term stability of the swelling properties.

### Example 3

### Protectants Protect Swelling Characteristics and Gel Integrity Following E-Beam Radiation

This study demonstrated that the network structure of samples was destroyed after radiation. However, the structure was well-maintained by the addition of protectants such as glycerin. It was demonstrated that the samples containing glycerin showed stable long-term data on their swelling characteristics while this data was not achievable for samples in all solutions in the absence of protectant because of the lack of the presence of robust network structure.

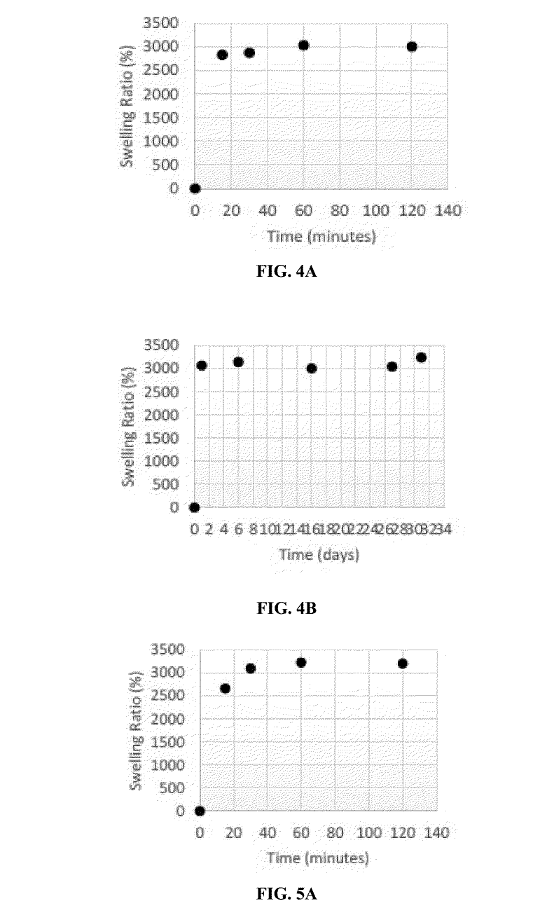

In the absence of glycerin, the maximal swelling ratio of a pure acrylamide gel reduced from about 3500% to about 3000% following low-temperature E-beam radiation (FIGS. 4A and 4B). Also, the radiation caused a loss of gel shape and integrity in the absence of glycerin. The gel sample was viscous and did not retain its shape following radiation.

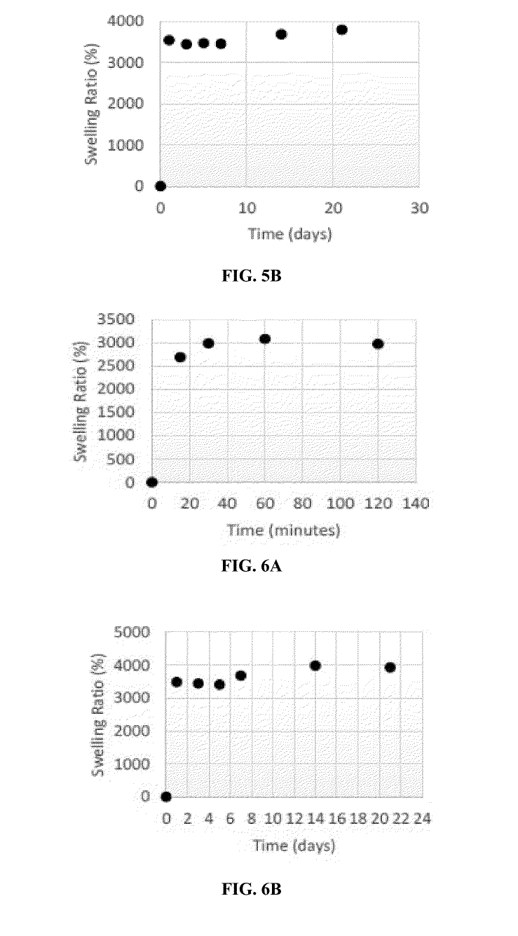

Addition of glycerin protected the swelling characteristics of the gels as well as gel integrity when the gels were treated with E-beam radiation. For example, addition of glycerin in a range of 0.1% to 1% restored the maximal swelling ratios to about 3500% when the acrylamide gels were swollen in human serum following low-temperature or ambient temperature E-beam radiation (FIGS. 5A-6B).

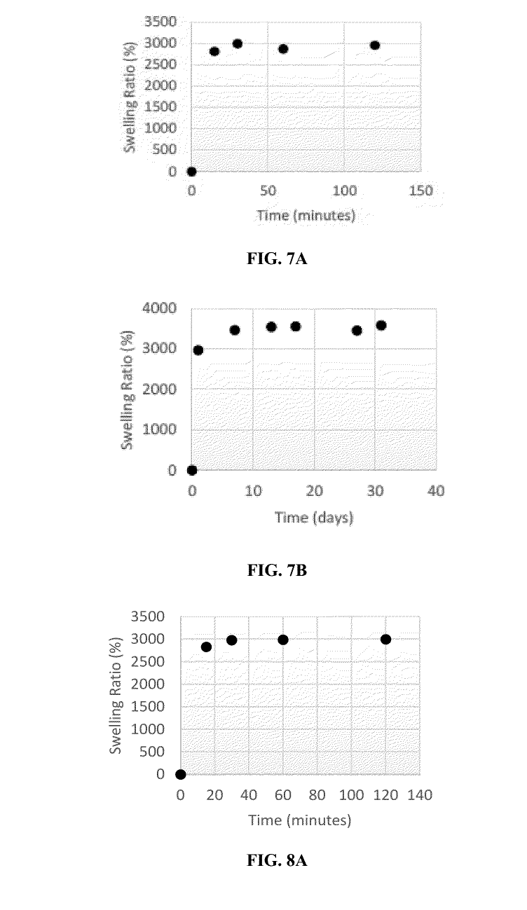

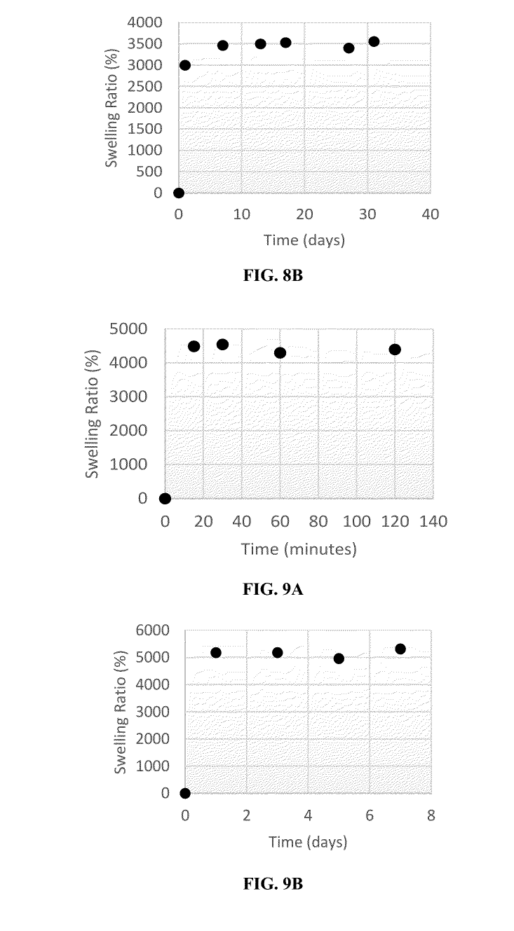

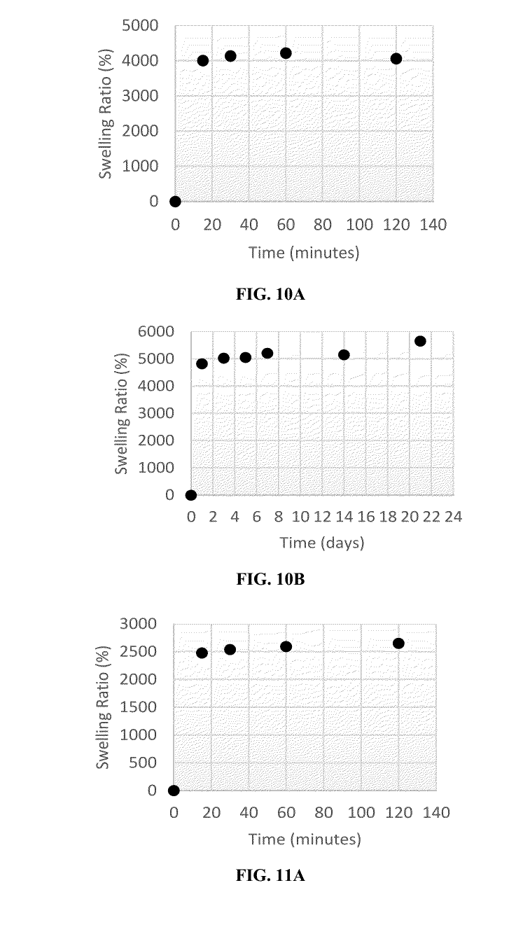

Similarly, addition of glycerin in a range of 0.1% to 1% restored the swelling ratio to about 3500% when the acrylamide gels were allowed to swell in bovine serum following low-temperature or ambient temperature E-beam radiation (FIGS. 7A-8B). Swelling in PBS of 0.1% to 1% glycerin-containing pure acrylamide gels achieved a swelling ratio of about 4000% during the initial 15 min of swelling, and up to 5000% during the long-term storage (FIGS. 9A-10B).

### Example 4

### Sterilization with Ethylene Oxide

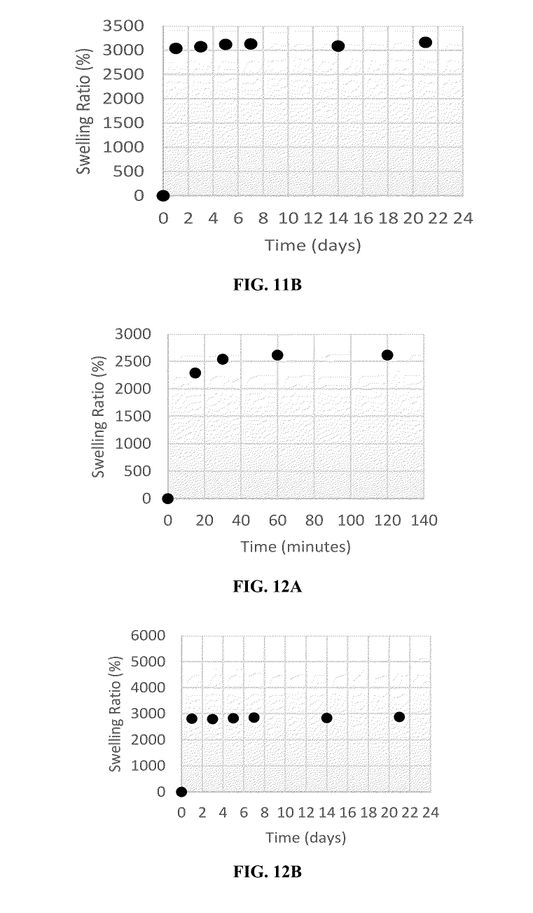

Sterilization of samples by ethylene oxide demonstrated that the samples maintained their structure after sterilization procedure and no adverse effect was observed. The swelling profile of samples in the first two hours and after three weeks is shown in FIGS. 11A-12B.

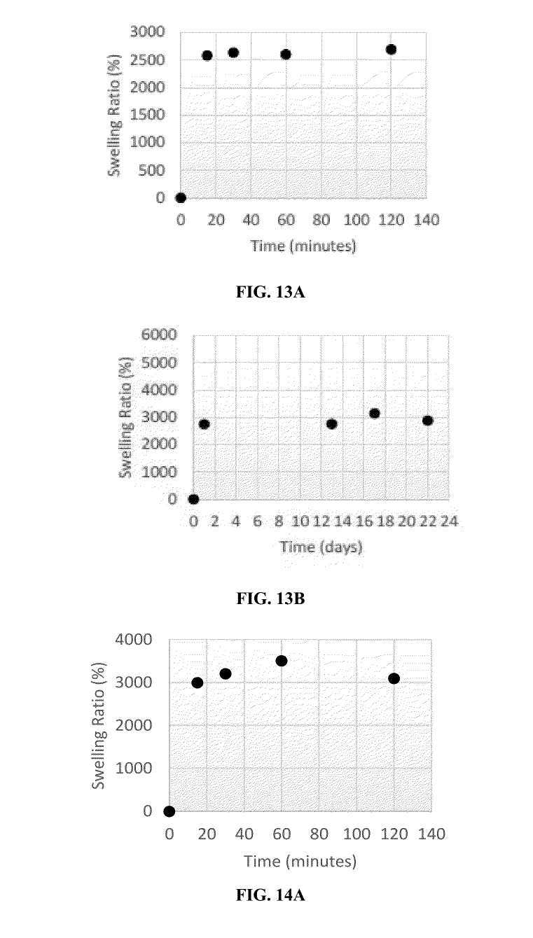

Following sterilization with ethylene oxide, the short-term swelling ratios of pure acrylamide gels were about 2500%, which later increased to 3000% when the gels were allowed to swell, as shown in FIGS. 11A and 11B. Addition of 0.1% to 1% glycerin did not change the swelling profile following ethylene oxide sterilization and swelling in human serum (FIGS. 12A-12B). The swelling ratios in bovine serum following sterilization with ethylene oxide were similar to those observed for swelling in human serum (FIGS. 12A-13B).

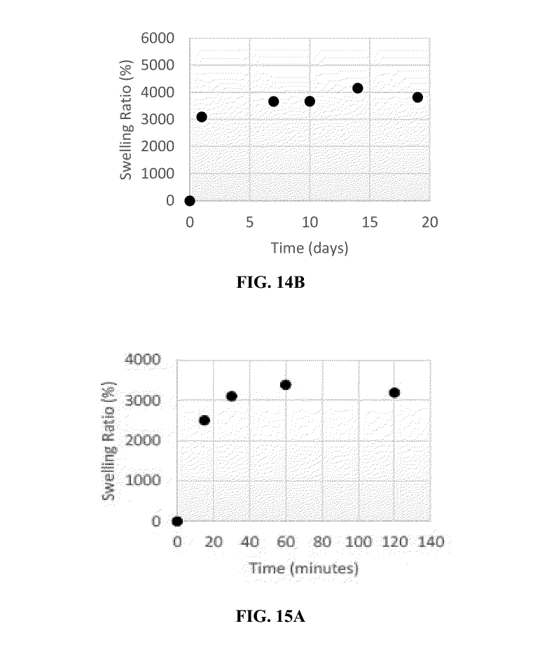

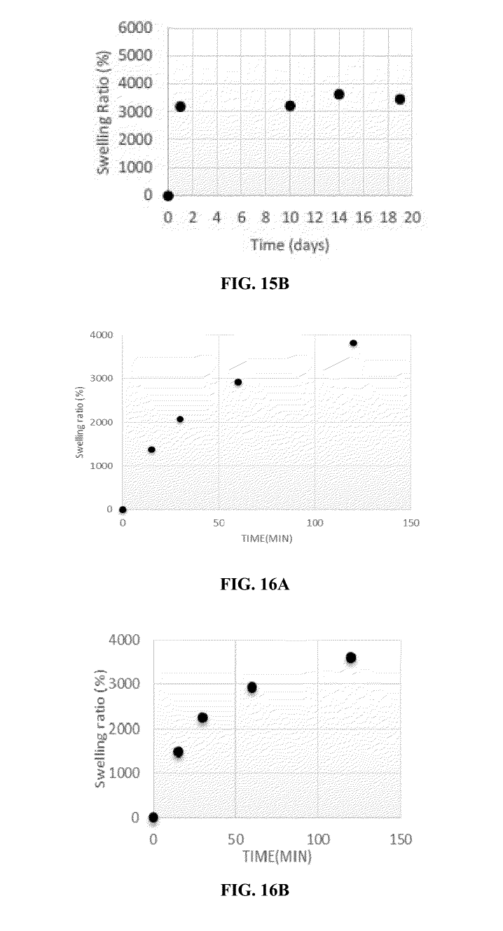

The swelling ratios of the pure acrylamide gels with or without glycerin in PBS were higher, and were about 3000% for short-term swelling, and 3500% for long-term swelling, following sterilization with ethylene oxide (FIGS. 14A-15B).

### Example 5

### Storage in Ethanol Solutions does not Affect the Thickness of Acrylamide Gels

Acrylamide gels were tested for their stability in ethanol solutions. The thickness of the gels before and after storage in ethanol solutions was evaluated and the results are shown in Table 2. Storage of acrylamide gels in 70% ethanol and 30% water solution did not affect the thickness of the gels. In other words, the gels did not swell when stored in 70% ethanol and 30% water solution.

TABLE 2
Sample thickness (μm) before and after storage in 70% ethanol.

| | Thickness | Thickness |
|---|---|---|
| | before storage | after storage |
| Solution | (μm) | (μm) |
| 70% ethanol + 30% H₂O | 130-140 | 120-150 |

The thickness of the gels before and after washing with ethanol solutions of various compositions was also tested and the results are shown in Table 3. The acrylamide gels retained their thickness, i.e. did not swell, when the gels were washed with ethanol solutions containing up to 30% water or 20% glycerin (Table 3).

TABLE 3
Sample thickness (μm) before and after
washing in different ethanol solutions.

| | Thickness | Thickness |
|---|---|---|
| | before washing | after washing |
| Solution | (μm) | (μm) |
| 100% ethanol | 140-160 | 140-160 |
| 80% ethanol + 20% Glycerin | 140-160 | 150-160 |
| 70% ethanol + 20% H₂O + | 140-160 | 130-160 |
| 10% Glycerin | | |
| 70% ethanol + 10% H₂O + | 140-150 | 100-110 |
| 20% Glycerin | | |
| 70% ethanol + 30% H₂O | 130-140 | 120-150 |
| 70% ethanol + 25% H₂O + | 170-180 | 140-160 |
| 5% Glycerin | | |

### Example 6

### Storage in Ethanol Solutions does not Affect the Swelling Characteristics of Acrylamide Gels

The swelling of acrylamide gels was tested before and after storage in different ethanol solutions. The swelling profiles of the acrylamide gels stored in ethanol were compared to those of not stored in ethanol solutions. The results are presented in FIGS. 16A and 16B and Table 4.

Similar swelling properties were observed for the gels stored in ethanol solutions containing between 70% and 100% ethanol (Table 4) in PBS. There was no difference in the swelling profile of acrylamide gels stored in 100% ethanol; in 80% ethanol and 20% glycerin; in 70% ethanol, 10% water and 20% glycerin; in 70% ethanol and 30% water; or in 70% ethanol, 25% water, and 5% glycerin.

TABLE 4
Swelling ratio (%) within initial 120 minutes of swelling in
PBS of pure acrylamide gels without (control) and with storage
in solutions containing between 70% and 100% ethanol.

| | | | | 70% | | 70% |
|---|---|---|---|---|---|---|
| | | | | ethanol | | ethanol |
| | | | 80% | 10% | 70% | 25% |
| | | | ethanol | water | ethanol | water |
| Time | | 100% | 20% | 20% | 30% | 5% |
| (min) | Control | ethanol | glycerin | glycerin | water | glycerin |
| 0 | 0 | 0 | 0 | 0 | 0 | 0 |
| 15 | 1065 | 802.7 | 1031.8 | 1027.4 | 1488.9 | 1123.3 |
| 30 | 1714 | 1758.6 | 2425 | 1880.2 | 2248.9 | 2016.5 |
| 60 | 2727 | 2576.6 | 2727.3 | 2683 | 2931.1 | 2709.7 |
| 120 | 3789 | 3435.1 | 3484.1 | 3312.3 | 3600 | 3400 |

### Example 7

### Pressing of Gels Reduces the Thickness of Dry Gels without Affecting Swelling Characteristics

The thickness of the gels in dry state was reduced by means of a calendering (mechanical rolling and pressing) in a consistent manner. This process reduced the thickness of the gels by about 50% or more from their original thickness. The results are shown in Table 5.

Also, the calendering process did not affect the swelling profile of the gels.

TABLE 5
Thickness (μm) of acrylamide and acrylic acid
gels before and after calendaring (pressing).

| | Thickness | Thickness |
|---|---|---|
| | before pressing | after pressing |
| | (μm) | (μm) |
| Acrylamide gels | 80-120 | 50-70 |
| Acrylamide gels | 180-240 | 150-200 |
| Acrylamide gels | 130-160 | 90-120 |
| Acrylamide gels | 90-140 | 80-110 |
| Acrylic acid gels | 200-330 | 150-290 |

### Example 8

### Composite Gels and their Properties

It is desirable that the hydrogel swells rapidly once the prosthesis is deployed so that the paravalvular leaks can be eliminated before the physician closes the case or before the final aortogram is performed. In order to achieve this, two different hydrogels, a combination of ionic and non-ionic hydrogels, were tested. The ionic hydrogels are denser and demonstrate faster swelling and have a lower viscosity as compared to the non-ionic hydrogel.

Strips of two types of gels, one formed from acrylamide monomer crosslinked with PEGDAA, and the other of acrylic acid monomers crosslinked with PEGDA, were combined in a capsule. These encapsulated composite gels showed improved swelling profile compared to those observed for either of the two gels separately.

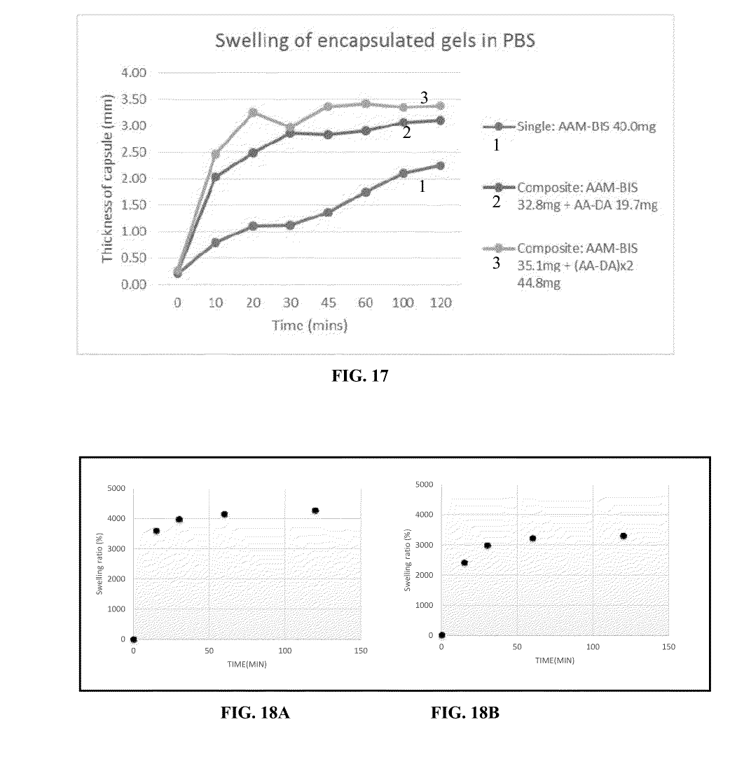

The swelling profiles of an encapsulated acrylamide gel (1) and of two encapsulated composite gels (2) and (3) are presented in FIG. 17. The gels were allowed to swell in PBS, and the thicknesses of the capsules were measured over time. The data showed that the thickness of capsules with composite gels increased faster and stabilized at a maximum thickness sooner than that for the capsule with a single gel.

The capsule with composite gel (2), encapsulating 32.8 mg of an acrylamide gel (AAM-BIS) and 19.7 mg of an acrylic acid gel (AA-DA), took 20 min to reach and stabilize at a maximum thickness of about 3.3 mm. The capsule with the composite gel (3), encapsulating 35.1 mg of an acrylamide gel (AAM-BIS) and 44.8 mg of an acrylic acid gel (AA-DA), took 30 min to reach and stabilize at the maximum thickness of about 3 mm. By contrast, the thickness of the capsule with a single gel (1) reached about 2.25 mm in 120 min. These data indicate that encapsulated composite gels swelled faster and stabilized sooner without loss of the absorbed fluid than encapsulated single gels. The AA-DA gels swell rapidly, whereas the AAM-BIS gels are stable at higher concentrations of ions, especially in solutions with multivalent positive ions. This property may be beneficial in areas where the stability of expandable hydrogels is desirable, such as during sealing of an endoluminal implant to a wall of a vessel.

### Example 9

### Copolymer Hydrogels

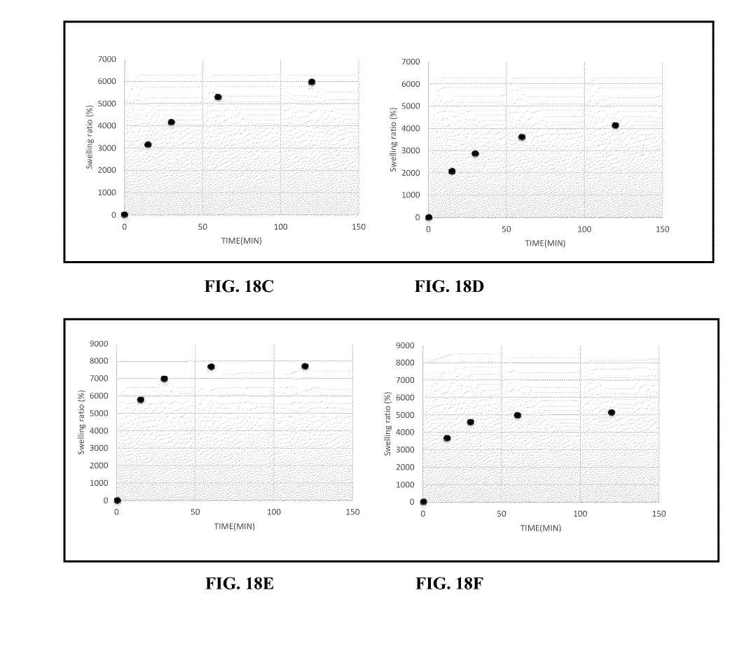

It has been found that gels made from a mixture of acrylamide (AAM) and acrylic acid (AA) monomers and a suitable cross linker such as N,N-methylene bisacrylamide (Bis) have properties that are better than gels made from either acrylamide and bisacrylamide or acrylic acid and PEGDA. The copolymer gels, typically containing acrylic acid in a range from 5-40% of the total monomer content (FIGS. 18A-18F), not only swell faster than acrylamide gels, but also swell significantly more—up to 60 to 70 times swell rate in PBS have been observed in comparison to 30-50% typically obtained for acrylamide or acrylic acid gels with similar monomer concentration. The copolymer gels are also stronger than equivalent gels made from either acrylamide or acrylic acid.

Another study of copolymer gel stability in serum was conducted and the results indicated that the composite gels do not show any signs of shrinkage in either bovine serum or calcium chloride solutions, whereas AA-DA gels show rapid shrinkage in both of these media. This finding is true for AAM/AA mixtures of 95/5, 90/10 and 80/20 ratios of the monomers. It is likely that the Ca²⁺ ions cause ionic cross-linking of the negatively charged carboxylic acid groups in the acrylic acid polymers. It is possible that a certain concentration or perhaps proximity of the AA groups is required for this cross-linking to take place.

Similarly, storage of AAM-Bis hydrogels and copolymer hydrogels with 80/20 ratios of the monomers in solutions containing ethanol does not change their dimensions, swelling and handling properties. These properties are summarized in Table 6 below.

TABLE 6
Hydrogel dimensions, thickness and handling properties
before and after storage in ethanol solution.

| | | Dimensions | Thickness | Handling |
|---|---|---|---|---|
| Sample | Storage | (mm) | (μm) | properties |
| AAm-Bis | Ethanol | 107.00 ± 10 | 160 ± 20 | Easy to handle |
| | solution | | | |
| Control | No storage | 107.12 ± 10 | 140 ± 20 | Easy to handle |
| AAm-Bis | | | | |
| Copolymer | Ethanol | 107.4 ± 10 | 100 ± 20 | Easy to handle |
| | solution | | | |
| Control | No storage | 103.3 ± 10 | 90 ± 20 | Easy to handle |
| copolymer | | | | |

Copolymer hydrogels also retain their swelling properties and stability when sterilized with E-beam, ethylene oxide (EO), or ethanol, or stored in ethanol/glutaraldehyde solution, PEG solution, or glycerin solution. The swelling ratios of copolymer gels with or without TEMED are presented in Table 7.

TABLE 7
Swelling ratios (%) of copolymer hydrogels following sterilization
or storage in water miscible organic solvents.

| | | | | Swelling Ratio² | | | | | |
|---|---|---|---|---|---|---|---|---|---|
| Storage/ | | Co- | | 0 | 15 | 30 | 60 | 1 | 21 |
| Steriliz | Condition | polymer¹ | Fluid | min | min | min | min | day | days |
| E-Beam | | | PBS | 0 | 5224 | 5458 | 5269 | 5197 | 5723 |
| | | | Serum | | 3745 | 3810 | 3815 | 3749 | 4003 |
| Ethylene | 55° C., | TEMED | PBS | 0 | 3080 | 2957 | 3022 | 3029 | 2930 |
| Oxide | 60% H³ | | | | | | | | |
| 750 mg/l | 37° C. | TEMED | PBS | 0 | 4269 | 4475 | 4585 | 4624⁴ | 4885 |
| | 40% H³ | | | | | | | | |
| | | | Serum | 0 | 3880 | 3846 | 3821 | 3639 | 4154⁶ |
| Ethylene | 55° C., | Without | PBS | 0 | 5716 | 5982 | 5655 | 5837 | 6366 |
| Oxide | 60% H³ | TEMED | | | | | | | |
| 750 mg/l | 37° C. | Without | PBS | 0 | 5693 | 6992 | 7665 | 7356⁴ | 7579 |
| | 40% H³ | TEMED | | | | | | | |
| | | | Serum | 0 | 4122 | 4928 | 5373 | 5368 | 5801⁶ |
| Ethanol/ | Control⁵ | | PBS | 0 | 5378 | 5815 | 5840 | 5677 | 6214 |
| Glut⁷ | 3 days | | PBS | 0 | 5803 | 6146 | 6006 | 5822 | 5983 |
| | 1 month | | PBS | 0 | 5620 | 5862 | 5880 | | |
| | 3 months | | PBS | 0 | 4426 | 4906 | 4961 | | |
| PEG | | | PBS | 0 | 5902 | 6702 | 6979 | | |
| Storage⁸ | | | | | | | | | |
| Glycerin | | | PBS | 0 | 5921 | 6825 | 6936 | | |
| Storage⁹ | | | | | | | | | |
| | | | DI | 0 | 91174 | 96944 | 94622 | | |
| | | | Water¹⁰ | | | | | | |

¹Copolymer gel containing 50% acrylic acid
²Swell Ratio (%) at indicated times following activation of copolymer hydrogel
³H—Humidity
⁴Value for day 2 of swelling
⁵Control—No storage or sterilization
⁶Value for day 26 of swelling
⁷Ethanol/Glut—Ethanol/Glutaraldehyde solution
⁸The PEG storage solution contained 80% PEG and 5M salt
⁹The glycerin storage solution contained 50% to 80% glycerin and 5M salt
¹⁰DI Water—Deionized water

The data in Table 7 show that short term (0-60 min) and long term (1-21 days) swelling ratio of copolymer hydrogels is not affected when swelling occurs in PBS or serum following sterilization of the copolymer hydrogels with E-beam, EO, or ethanol.

The gels maintained their structural integrity over 30 days after E-beam sterilization.

The temperature and humidity were varied during EO sterilization, and the gels showed a minimal reduction in swelling ratio under these conditions. The presence of TEMED results in the esterification of copolymers after exposure to EO. Elimination of TEMED from the synthesis procedure of the copolymer gels prevented the reduction in swelling ratio of gels. Other parameters in synthesis (e.g. initiators) were modified to obtain a robust gel in the absence of TEMED. For example, other initiators were tested at concentrations of between 0.0002 g/ml and 0.01 g/ml.

Copolymer gels stored for 3 days or one month in ethanol showed no change in swelling ratio when compared to that of no-storage control. However, the swelling ratio of gels decreased after storing in the ethanol solution for 3 months.

Also, storing the copolymer hydrogel in non-alcoholic solutions such as PEG and glycerin solutions, had no impact on the swelling ratio of the gels. The swelling ratio of the copolymer hydrogels in DI water is shown for comparison.

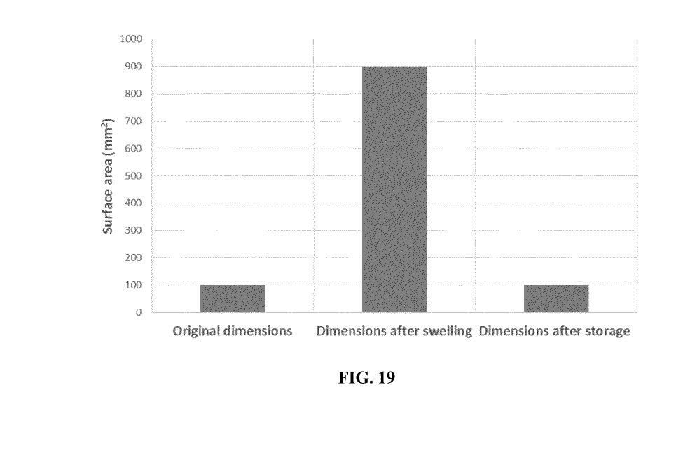

In addition, the copolymer hydrogels are able to regain their pre-swelling size when transferred into storage solution following swelling. FIG. 19 demonstrates the change in surface area (mm²) of a copolymer hydrogel from its surface area in dry state, to its surface area after swelling in PBS, and recovery of its original surface area when the swollen copolymer hydrogel is placed in a storage solution. The storage solution contained between 50% and 100% poly(ethylene) glycol and between 0.5 M and 5 M sodium and phosphate salts.

### Example 10

### Hydrogels with Better Swelling Ratio and Strength (High Porosity)

It has been found that gels made in a way that encourages formation of larger pore sizes while maintaining overall monomer and cross-linker concentrations and ratios have properties that are superior to gels made in a conventional fashion. Such gels have better swelling properties and have higher breaking strength. These gels can be made from a number of monomers, including acrylamide and acrylic acid and suitable cross linkers such as N,N methylene bisacrylamide or PEG DA or PEGDAA.

These gels are made by adding high concentrations of salt, solvents or other chaotropic agents to the reaction mixture during polymerisation. Salts, solvents and other chaotropic agents that can be utilized include, but are not limited to, Sodium chloride, potassium chloride, Sodium sulphate, ammonium sulphate, magnesium chloride, guanidinium hydrochloride, thiourea, urea, methanol, ethanol, isopropanol, 1-propanol, butanol, acetone, dimethyl sulfoxide.

Ideally the polymerisation reaction remains in a single phase, but in some cases the reaction mixture may form a closely intertwined network of two or more phases. It is theorized that the addition of these agents cause the polymer chains to form “bundles” that leave bigger pores in the gel than if the polymers were formed without addition of significant amounts of these agents. Such bundling in some cases causes the resulting gel to have a higher strength than a gel made by a conventional method.

### Example 11

### Additional Hydrogel Formulations Suitable for Use in Seals

As the ideal gel has rapid swelling and reaches its maximum swelling state quickly, the desired gels consist of 15% Acrylic acid and 0.05% poly(ethylene glycol) diacrylate, or of 10% Acrylic acid and 0.05% poly(ethylene glycol) diacrylate. Such suitable gels are presented in Publication Nos. US 2013/0331929, US 2013/0190857, and US 2013/0197622.

Assessment of Alternative Crosslinkers for Hydrogels

The principle behind the selected crosslinkers is that rather than having a short cross-linker with only two polymerizable groups, a polyvalent crosslinker (i.e., a long-chain hydrophilic polymer with multiple polymerizable groups) is being used. A much stronger hydrogel is obtained compared to short chain, divalent crosslinkers. While these gels are very firm, they possess very good swelling characteristics. Very strong gels do not normally swell very much.

Poly vinyl alcohol (PVA) was derivatized with allyl glycidyl ether under alkaline conditions. Gels were made by combing acrylic acid with the PVA-based crosslinker and then polymerizing the mixture by free radical polymerization using ammonium persulfate and TEMED as initiators.

In principle, the crosslinker can be made with a number of different starting materials: A range of PVAs as well as partially hydrolyzed poly vinyl acetates, 2-hydroxyethyl methactylates (HEMA) or various other polymers with reactive side groups can be used as the basic polymeric backbone. In addition, a wide range of natural hydrocolloids such as dextran, cellulose, agarose, starch, galactomannans, pectins, hyaluronic acid etc. can be used. A range of reagents such as allyl glycidyl ether, allyl bromide, allyl chloride etc. can be used to incorporate the necessary double bonds into this backbone. Depending on the chemistry employed, a number of other reagents can be used to incorporate reactive double bonds.

Preparation of Polyvalent Crosslinker

Polyvinyl alcohol (PVA, 30-70 kDa) was derivatized with allyl glycidyl ether under alkaline conditions. 2 g PVA was dissolved in 190 mL water. Once fully dissolved, 10 mL 50% NaOH was added, followed by 1 mL allyl glycidyl ether and 0.2 g sodium borohydride. The reaction was allowed to proceed for 16 hours. Subsequently, the crosslinker was precipitated from the reaction mixture by addition of isopropanol. The precipitate was collected by filtration, washed with isopropanol, and re-dissolved in 50 mL of water. The crosslinker was utilized for gel formation, as described below without further purification or characterization.

Gel Formation and Characterization

Gels were formed by combining acrylic acid with the PVA-based crosslinker prepared above, and then polymerizing the mixture by free radical polymerization using ammonium persulfate and TEMED as initiators.

Three gels were prepared containing 15% acrylic acid in combination with various ratios/concentrations of the PVA-based crosslinker. The components listed in Table 8 (excluding initiators) were mixed and degassed by placing the tubes in a desiccator with a vacuum applied. After 10 minutes, the vacuum was turned off, and the tubes remained in the desiccator for a further 10 minutes under vacuum. The desiccator was opened, and the initiator was added. The contents of the tubes were then mixed thoroughly. The tubes were capped and left overnight to polymerize, forming hydrogels. The swelling properties of the gels are presented in Table 9.

TABLE 8
Composition of gels 23a-c formed using
polyvalent PVA-based crosslinkers.

| Components | Gel | | |
|---|---|---|---|
| (mL) | 23a | 23b | 23c |
| acrylic acid | 1.5 | 1.5 | 1.5 |
| PVA cross-linker | 0.0526 | 0.526 | 5.26 |
| 50% NaOH | 1.251 | 2.15 | 2.35 |
| H2O | 7.122 | 5.779 | 0.795 |
| APS | 0.04 | 0.04 | 0.04 |
| TEMED | 0.05 | 0.05 | 0.05 |
| total | 10.02 | 10.05 | 10.00 |
| pH (pre-initiator addition) | 7.416 | 7.557 | 7.451 |

TABLE 9
Swelling behavior of gels 32a-c formed using
polyvalent PVA-based crosslinkers.

| Gel | 23a | 23b | 23c |
|---|---|---|---|
| 5 min swelling* | 1000-2000% | 250-1100% | 900-1000% |
| 60 min swelling* | 4000-6000% | 1100-2500% | 3600-4300% |

*3 repeats were made for each gel swelling experiment

### Example 12

### Sealing of a Type II Endoleak with Endoluminal Devices Containing Expandable Hydrogels

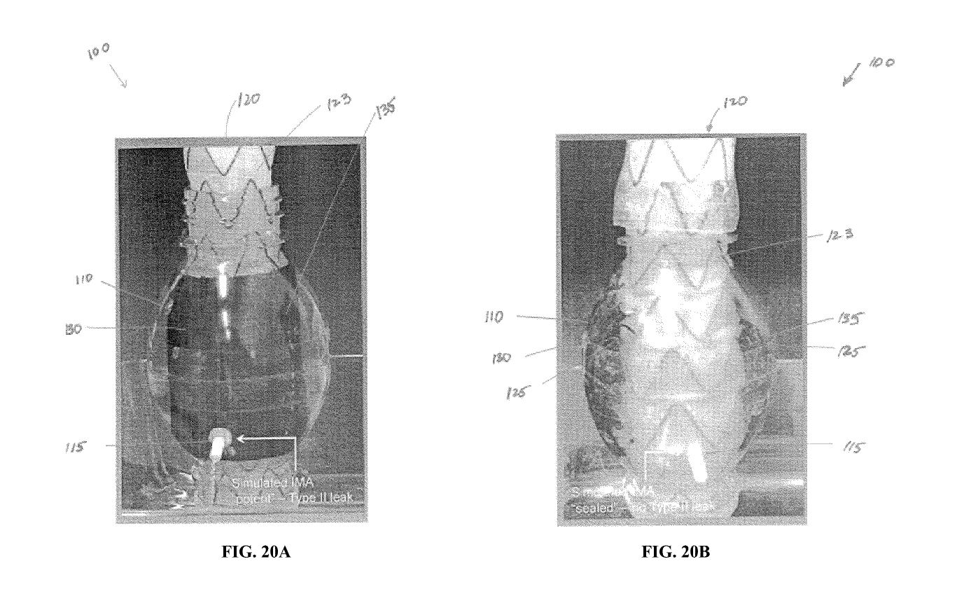

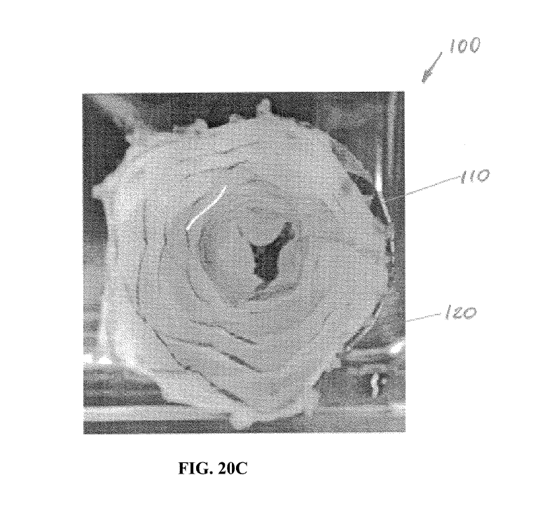

FIGS. 20A and 20B are prospective views of a replica aneurysm-device assembly 100. The assembly 100 included a replica of a 7 cm diameter aneurysm 110 and an endoluminal device 120 (FIG. 20A). The endoluminal device 120 was placed within the lumen of the aneurysm 110 at about 10% oversizing. The endoluminal device 120 included a 32 mm stent graft 123 and an expandable hydrogel 125 (not visible prior to expansion) deposited in dry/unhydrous state on the outside of the stent graft 123. The walls of the replica aneurysm 110 were transparent. The stent graft 123 was covered with vertically overlapping sheets 130 and 135 to demonstrate the swelling of the hydrogel. At time=0 min, the potent type II endoleak 115 released a stream of fluid. The hydrogel was then allowed to swell in water, for 20 min, at room temperature. At approximately 20 min, the simulated type II endoleak 115 was sealed by the expanded hydrogel 125 (FIG. 20B), there was no longer a stream of fluid escaping from the aneurysm 110. As the gel expanded, the sheets 130 and 135 were pushed outwards exposing the expanded hydrogel 125 of the endoluminal device 120. FIG. 20C is a plan view of the aneurysm-device assembly 100 as viewed from the top of the replica assembly 100.

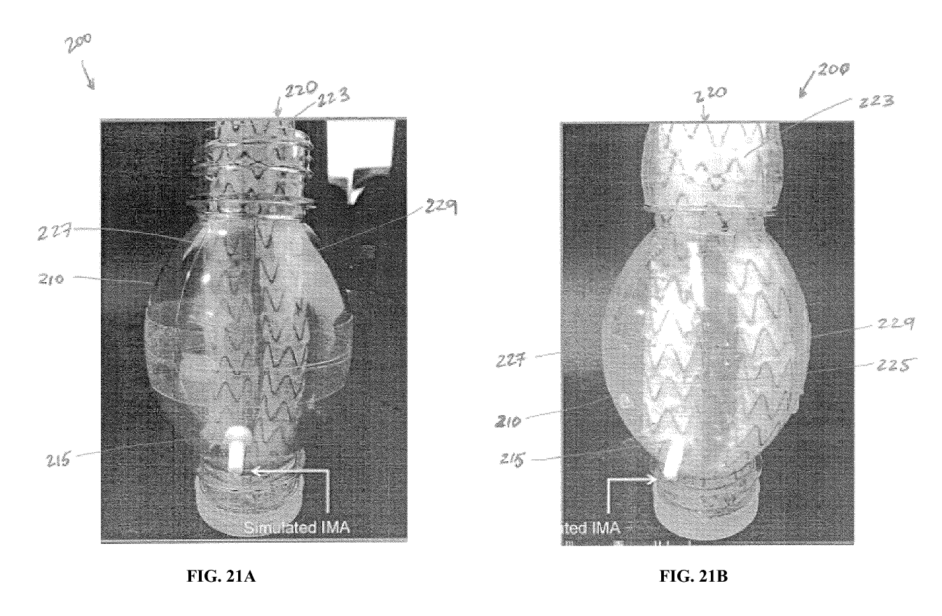

In another embodiment, the stent graft has a main body and a contralateral limb, and the hydrogel is deposited only on the contralateral limb. FIGS. 21A and 21B are perspective views of such a stent graft, and demonstrate an aneurysm-device assembly 200 simulating sealing of a type II endoleak 215. The assembly 200 included a replica of a 7 cm diameter aneurysm 210 and an endoluminal device 220. The endoluminal device 220 included a 32 mm stent graft 223 having a main body 227 and a contralateral limb 229. The device 220 also included an expandable hydrogel 225 (not visible prior to expansion) deposited in dry/unhydrous state on the outside of the contralateral limb 229. The device 220 was placed within the lumen of the aneurysm 210 at about 10% oversizing. The walls of the replica aneurysm 210 were transparent. At time=0 min there was a potent type II endoleak 215 (FIG. 21A). The hydrogel was then allowed to swell in water, for 20 min, at room temperature. At approximately 20 min (FIG. 21B), the simulated type II endoleak 215 was sealed by the expanded hydrogel 225; there was no longer a stream of fluid escaping from the aneurysm 210. This example demonstrated that expandable hydrogels deposited on only the contralateral limb of the stent graft may expand and seal a type II endoleak without changing the main body of the stent graft.

In another embodiment the hydrogel is hydrated by using a port connected to the contralateral limb (or the stent graft) at the section where the hydrogel is present. An externally injected fluid, e.g. sterile saline, deionized water, etc. may be used to hydrate the hydrogel through that port. This is particularly helpful when extreme expansion of the hydrogel is required e.g. for filling the complete aneurysm sac. Such an embodiment allows for controlled expansion of the hydrogel while it is still fully encapsulated within a membrane (impermeable and/or semipermeable). Once the required expansion is obtained the external connection to the port may be disconnected. The port in this embodiment serves as a one-way port.

### Example 13

### Sealing Endoleaks in Complex Abdominal Aortic Aneurysm (AAA)

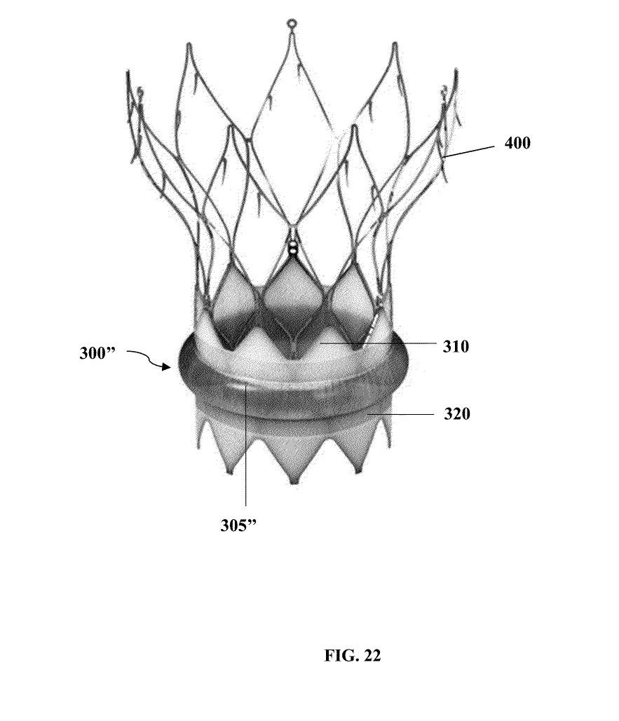

FIGS. 22-26 demonstrate how the highly expandable hydrogels can be applied in a seal to seal endoleaks in complex AAA.

FIG. 22 is a drawing showing the seal 300 in expanded state 300″ with an inner semi-permeable membrane 310 for controlled activation of the hydrogel, an outer membrane 320 for controlled expansion and complete encapsulation of the hydrogel 305, which is in expanded state 305″, and flared proximal bare stent 400. The sealing device is compatible with current generation stent graft systems and chimneys for use in the ChEVAR.

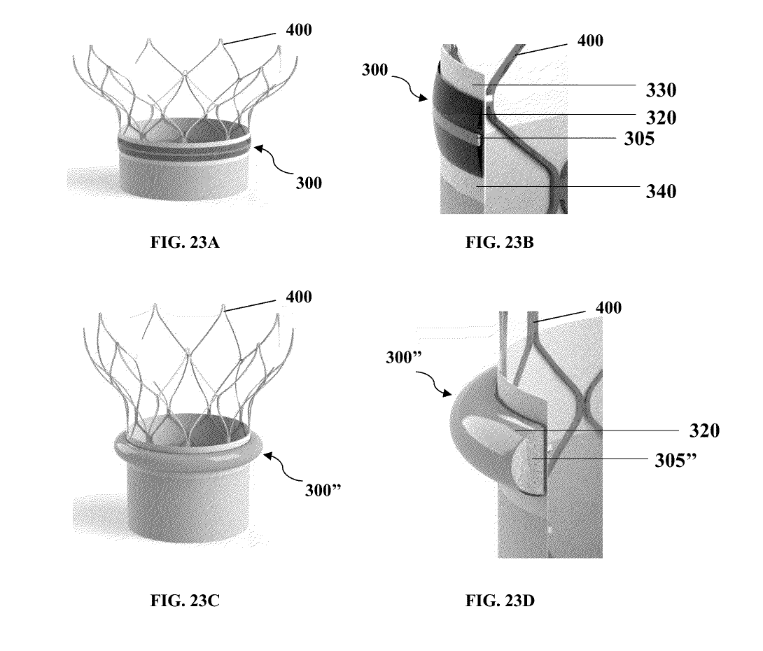

FIGS. 23A and 23B are drawings showing the seal 300 in an unexpanded state attached to a stent graft 400. FIG. 23A is a three-dimensional front view and FIG. 23B is a three-dimensional cross-sectional view of the seal 300 attached to the stent graft 400. In a cross-sectional view the seal 300 is attached to a thin graft material 330, a standard graft material 340, and holds the dry hydrogel 305 in a capsule 320. FIGS. 23C and 23D are drawings showing the seal 300 in an expanded state 300″ with the swollen hydrogel 305″ attached to a stent graft 400.

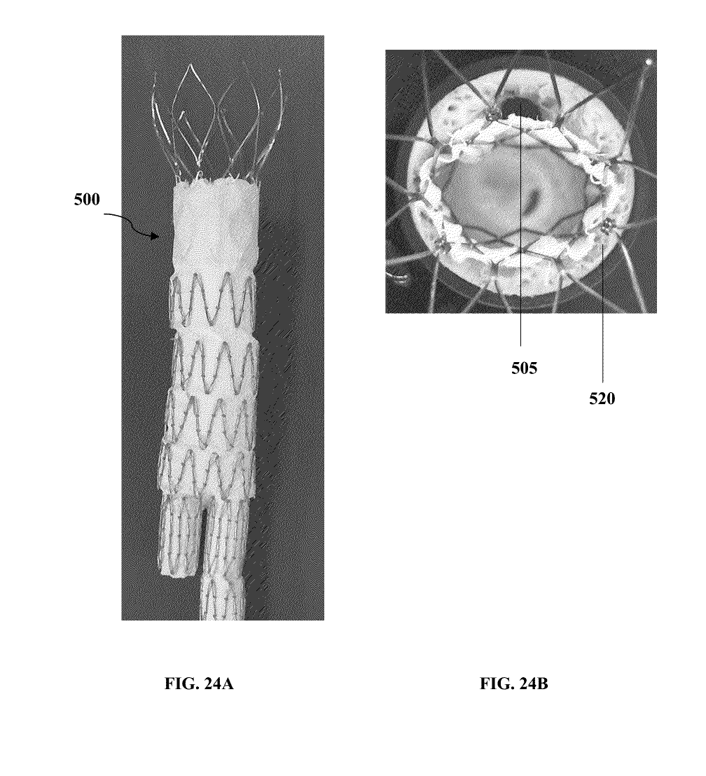

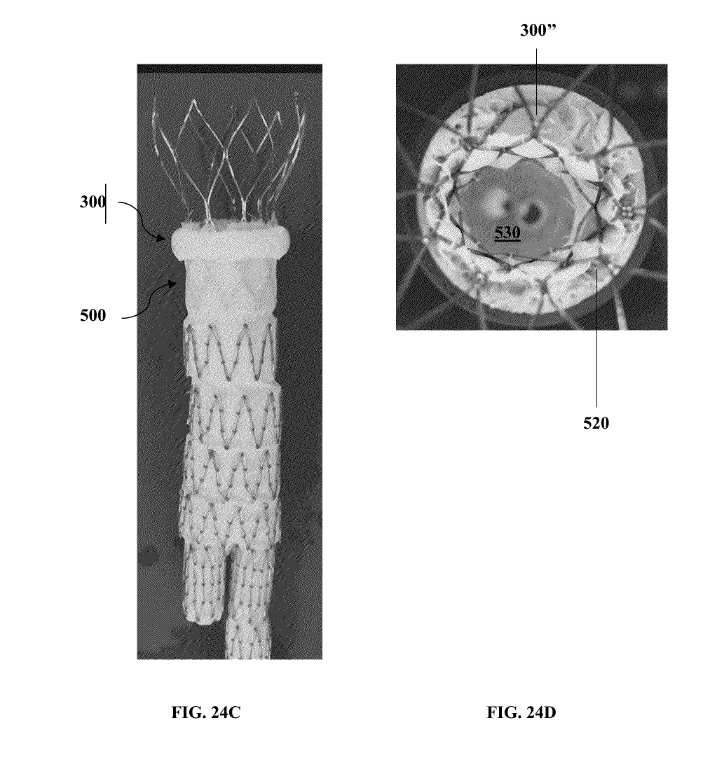

FIGS. 24A and 24B are drawings showing a bifurcated stent graft 500 without the seal. When in use, such a graft generates a leak site 505 due to incomplete apposition of the stent graft 500 to a heavily thrombotic vessel wall 520 (FIG. 24B). FIGS. 24C and 24D are drawings showing a bifurcated stent graft 500 with an expandable seal 300. Inclusion of the seal 300 onto the bifurcated stent graft 500 prevents the leak site from forming, as the seal 300 is highly conformable and remains securely positioned following expansion 300″, and the leak site 505 is eliminated by the highly conformable seal 300 without causing any deformation to the open central orifice 530 of the stent graft 500 (FIGS. 24C and 24D).

The seal can be used with flared proximal bare laser cut stent, or without the proximal bare stent. Therefore, the seal can be used with any suitable EVAR device with diverse stent designs. One such device is Chimney EVAR, or ChEVAR.

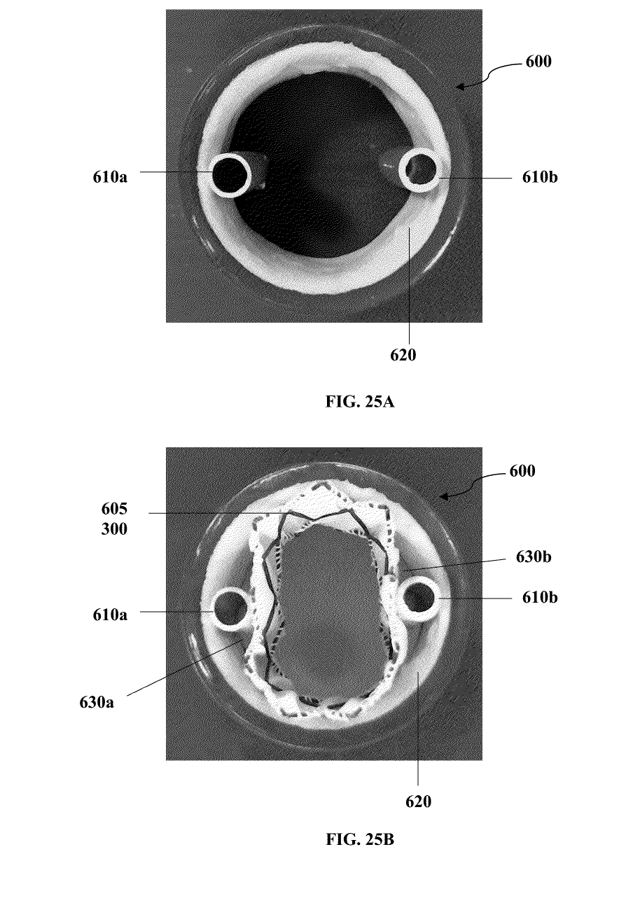

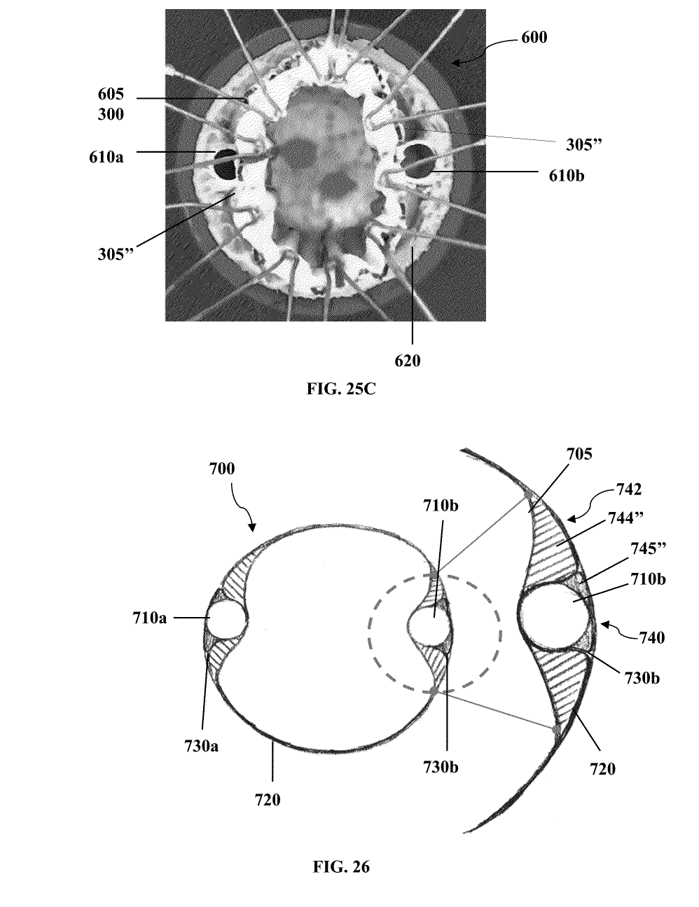

FIGS. 25A-25C are drawings showing the septs in a simulated sealing of gutter leak sites 630a and 630b with the seal 300 during implantation of a ChEVAR device 600. FIG. 25A demonstrated deployment of simulated chimney stents 610a and 610b against a simulated aortic wall 620 as step 1 of the simulated sealing. The gutter leak sites 630a and 630b are present following deployment of the ChEVAR device 600 with the stent graft 605 with seal 300 but before activation of the seal hydrogel (FIG. 25B, step 2 of the simulated sealing). Activation of the seal 300 causes swelling of the hydrogel 305 and formation of the expanded hydrogel 305″, which seals and eliminates the gutter leak sites 630a and 630b (FIG. 25C).

FIG. 26is a drawing showing the seal further minimizing gutter leaks if present on both of the chimneys and the stent graft of the ChEVAR device 700 with chimney stents 710a and 710b. The enlarged segment on the right demonstrates a chimney 710b with a chimney seal 740 and a stent graft 705 with a graft seal 742. The activated hydrogel 745″ of the chimney seal 740 and the activated hydrogel 744″ of the stent graft seal 742 together further minimize the leak sites 730a and 730b along the arterial wall 720. FIG. 26 demonstrates that the seal can be present on both the chimneys of the ChEVAR device, and the stent graft. Using such a device, the gutter leak sites can be further minimized, as shown in the enlarged segment on the right.

We claim:

1. A hydrogel comprising
a polymer selected from poly(acrylic acid), poly(acrylamide), and poly(metacrylic acid), copolymers and blends of each,
crosslinked with a di- or polyvalent crosslinking agent,
wherein the hydrogel does not swell in storage or sterilization solution,
wherein the hydrogel swells in aqueous fluid 200 to 1000 fold the weight of its dry state in less than about 15 minutes, and
wherein the weight of the swollen hydrogel remains unchanged over time when the hydrogel is in the aqueous fluid.

2. The hydrogel of claim 1, wherein the polyvalent crosslinking agent is selected from the group consisting of bis-acrylamide or di-acrylamide, di(ethylene glycol) diacrylate, poly(ethylene glycol) diacrylate, and long-chain hydrophilic polymers with multiple polymerizable groups.

3. The hydrogel of claim 1, further comprising a protectant.

4. The hydrogel composition of claim 3, wherein the protectant is glycerin.

5. The hydrogel composition of claim 3, wherein the protectant is ascorbic acid or trehalose.

6. The hydrogel composition of claim 1, further comprising an agent selected from the group consisting of prophylactic, therapeutic, diagnostic, and imaging agents.

7. The hydrogel composition of claim 6, wherein the therapeutic agent is a blood-clotting agent.

8. The hydrogel composition of claim 1, wherein the hydrogel retains its swelling characteristics after storage in the storage or sterilization solution.

9. The hydrogel composition of claim 1, wherein the hydrogel retains its swelling characteristics and does not swell when sterilized with ethylene oxide or radiation using electron bean or gamma radiation.

10. The hydrogel of claim 1, wherein the hydrogel retains its swelling characteristics after calendering.

11. The hydrogel of claim 1, wherein the polymer is a composite polymer comprising any combination of poly(acrylic acid), poly(acrylamide), and poly(metacrylic acid), and copolymers and blends of each.

12. An endoluminal seal for sealing an endoluminal implant or prosthesis to a wall of a lumen of a subject, the endoluminal seal comprising:
an expandable material selected from the group consisting of hydrogels, sponges and foams optionally spray dried or chemically coupled to the interior of the endoluminal seal, and
a first membrane adjacent to and containing the expandable material;
wherein the expandable material is activated by exposure to a fluid or a foaming agent, and
wherein the first membrane is semi-permeable, optionally comprising access port/means to allow for an aqueous media to be inserted externally that hydrates the gel.

13. The endoluminal seal of claim 12, wherein the hydrogel comprises
a polymer selected from poly(acrylic acid), poly(acrylamide), and poly(metacrylic acid), copolymers and blends of each,
crosslinked with a di- or polyvalent crosslinking agent,
wherein the hydrogel does not swell in storage or sterilization solution,
wherein the hydrogel swells in aqueous fluid 200 to 1000 fold the weight of its dry state in less than about 15 minutes, and
wherein the weight of the swollen hydrogel remains unchanged over time when the hydrogel is in the aqueous fluid.

14. The endoluminal seal of claim 12 that is positioned within or is close abutment to the exterior of the implant or prosthesis, not changing the profile from that of the implant or prosthesis during implantation.

15. The endoluminal seal of claim 12 that expands under sufficient low pressure so that it seals the space between the implant or prosthesis and luminal wall, but does not push the implant or prosthesis away from the lumen wall.

16. The endoluminal seal of claim 12, wherein the seal actively conforms to a leak site between the lumen wall and the implant or prosthesis, without altering the rest of the device configuration.

17. The endoluminal seal of claim 12 comprising a support member which interfaces between the seal and the endoluminal implant or prosthesis and can go from an unexpanded or crimped state to an expanded state.

18. The endoluminal seal of claim 17, wherein the support member is an expandable mesh or struts, optionally including means for securing the implant or prosthesis at the site of implantation.

19. The endoluminal seal of claim 12, wherein the seal is crimped distal or proximal to the prosthesis, and aligned with the prosthesis prior to or at the time of placement.

20. The endoluminal seal of claim 12, further comprising an adhesive.

21. The endoluminal seal of claim 12 being storable in ethanol, preferably in an ethanol solution containing ethanol at a concentration ranging from between 30% and 100%, and being washable with the ethanol solution, preferably between 0 to 4° C.

22. The endoluminal seal of claim 21 having been stored or washed in ethanol before it is introduced within the body.

23. A method of sealing a lumen comprising implanting an endoluminal implant or prosthetic comprising one or more of an endoluminal seal comprising:
an expandable material selected from the group consisting of hydrogels, sponges and foams optionally spray dried or chemically coupled to the interior of the endoluminal seal, and
a first membrane adjacent to and containing the expandable material;
wherein the expandable material is activated by exposure to a fluid or a foaming agent, and
wherein the first membrane is semi-permeable, optionally comprising access port/means to allow for an aqueous media to be inserted externally that hydrates the gel,
wherein the endoluminal seal can be affixed thereto into a wall of a lumen of a subject.

24. An endoluminal device comprising
a stent graft with a lumen, and
an endoluminal seal of comprising:
an expandable material selected from the group consisting of hydrogels, sponges and foams optionally spray dried or chemically coupled to the interior of the endoluminal seal, and
a first membrane adjacent to and containing the expandable material;
wherein the expandable material is activated by exposure to a fluid or a foaming agent, and
wherein the first membrane is semi-permeable, optionally comprising access port/means to allow for an aqueous media to be inserted externally that hydrates the gel on the outside of the stent,
wherein the seal is positioned in abutment with the stent,
wherein the seal seals the device to a vessel wall, and
wherein the seal and the stent allow blood flow through the lumen of the device.
